(19) United States
(12) Patent Application Publication
(10) Pub. No.: US 2026/0130880 A1
(43) Pub. Date: May 14, 2026

(54) DIGITAL THERAPEUTICS AND COMBINATION THERAPY FOR THE TREATMENT OF PARKINSONS DISEASE

(71) Applicant: REMEPY HEALTH LTD, Ramat Hasharon (IL)

(72) Inventors: AMIR AMEDI, Modiin-Macabim Reut (IL); OR SHOVAL, Ramat Hasharon (IL); Michal TSUR-SHALEV, Zichron Yaacov (IL); NIRA NEOMI SAPORTA, Givatayim (IL); SHAHAR SHELLY, Herzlyia (IL); RENAN GUTMAN, Hoboken, NJ (US)

(21) Appl. No.: 19/385,552

(22) Filed: Nov. 11, 2025

Related U.S. Application Data
(60) Provisional application No. 63/718,766, filed on Nov. 11, 2024.
(60) Provisional application No. 63/780,722, filed on Mar. 31, 2025.

(51) Int. Cl. A61K 31/198; A61B 5/00; A61K 31/192; A61P 25/16

(52) U.S. Cl. CPC A61K 31/198; A61B 5/4082; A61B 5/4848; A61K 31/192; A61P 25/16

(57) ABSTRACT

Provided is a method of digital therapeutics for modulating one or more Parkinson's disease-related markers, symptoms, or disease progression in an individual, on a standalone basis or by combining them with medicinal agents, particularly dopaminergic agents. Also provided are corresponding systems for implementing, delivering, and adapting such therapeutic interventions.

### CROSS REFERENCE TO RELATED APPLICATIONS

This application claims the benefit of U.S. Provisional application No. 63/718,766, filed on Nov. 11, 2024, and U.S. Provisional application No. 63/780,722 filed on Mar. 31, 2025, which is/are incorporated in their entirety herein by reference.

### FIELD OF THE INVENTION

The presently disclosed subject matter relates to the field of digital therapeutics for modulating one or more Parkinson's disease-related markers, symptoms, or disease progression in an individual, on a standalone basis or by combining them with medicinal agents, particularly dopaminergic agents.

### BACKGROUND

Parkinson's disease (PD), the second most common neurodegenerative disorder, affects millions worldwide and is a growing global health challenge. While levodopa remains the cornerstone of treatment, its long-term use is limited by motor complications and its little effect on non-motor symptoms (NMS), resulting in significant unmet needs in effectively managing both motor and non-motor aspects of PD.

Multimodal non-pharmacological therapies, including physical, cognitive, and psychological interventions, are recognized as essential to comprehensive PD management. While multidisciplinary approaches improve both motor and NMS, access remains limited even in high-income countries. Despite clinical guidelines, the utilization of non-pharmacological interventions remains low due to geographic, financial, logistical, and public health educational barriers, a gap that digital health could address, but has yet to efficiently integrate into routine care. Currently, most PD digital solutions narrowly focus on motor symptom tracking (e.g., gait and balance) without targeting improvement, and with limited integration of validated psychological or cognitive strategies for NMS. Additionally, very few digital platforms are personalized or have undergone rigorous evaluation of treatment efficacy or underlying mechanisms, partly due to methodological challenges such as developing placebo controls for digital therapeutics.

In addition to exploring therapeutic benefits, present disclosure discloses neural correlates of improvement, focusing on the thalamus as a key integrative hub within basal ganglia-thalamocortical circuits that regulate motor, cognitive, and limbic functions. Neurodegeneration across these parallel thalamocortical circuits contributes to the core motor and NMS of PD. Furthermore, the thalamus's involvement in both motor and NMS highlights its central role in PD-related circuit pathology and its potential as a target for therapeutic modulation. Notably, rs-fMRI is sensitive to treatment-induced plasticity: levodopa increases functional connectivity in the ON-state compared to the OFF-state or drug-naïve conditions. Additionally, rehabilitation targeting motor, cognitive, or affective functions can further modulate network dynamics. However, existing longitudinal studies often assess motor and non-motor domains separately, rarely probing specific thalamocortical subcircuits.

To address the therapeutic gap for scalable and multidisciplinary interventions, the present invention involves a digital therapeutic application designed to augment standard dopaminergic treatment, but also on a stand-alone basis for disease modulation. The sensorimotor-deprivation component included synchronized visual and auditory stimulation, sensory substitution and deprivation/inhibition, and adaptive gamification via spatial memory tasks. These cognitive neuroscience elements were shown to enhance circuit-level plasticity, and were complemented by psychological strategies targeting emotional regulation. Together, they reduced depressive symptoms and modulated rsFC across cognitive, affective, and visuomotor networks, including limbic regions such as the amygdala and medial temporal lobe (MTL). These same regions are critically implicated in PD, where structural and functional alterations are consistently linked to non-motor symptoms. Adapting this framework, the PD-specific version also incorporated daily modules targeting additional sensorimotor activations, speech, motor, and emotion regulation aspects. The present disclosure supports and extends the therapeutic effect of (e.g.,) levodopa by engaging brain circuits that pharmacological treatment alone may not fully activate and to identify links between usage patterns and outcomes.

The use of a multi-modality therapeutic approach offers several key advantages that enhance both clinical effectiveness and user experience. By integrating diverse intervention types, such as cognitive tasks, physical exercises, voice training, and relaxation modules, the system leverages synergistic effects, where improvements in one domain can positively influence others (e.g., enhanced sleep supporting better motor performance). This cross-modal interaction enables more precise and dynamic optimization of treatment plans, tailored to the evolving needs of the individual. Additionally, multi-modality increases accessibility by accommodating different user abilities, preferences, and engagement styles. It also facilitates comprehensive data synchronization across modalities, allowing for richer insights and more responsive adaptation of the therapeutic protocol over time.

### SUMMARY

In one embodiment the invention provides a method for modulating one or more Parkinson's disease-related markers, symptoms, or disease progression in an individual in need thereof, the method comprising:
determining a therapeutic plan to be delivered to the individual, wherein the therapeutic plan comprises therapeutic interventions selected from: at least one motor skills intervention, at least one psychological intervention, at least one cognitive intervention, or a combination thereof;
converting the therapeutic plan into a plurality of targeted interaction outputs for presentation to the individual; and
adaptively delivering the therapeutic plan, tracking the individual's responses and associated performance metrics, and continuing until a predetermined condition is met.

In one embodiment the method further comprises obtaining a plurality of attributes associated with the individual from a plurality of data acquisition sources to generate an initial profile for the individual, wherein the determining of the therapeutic plan further comprises adapting the therapeutic plan based on the initial profile.

In one embodiment the therapeutic plan comprises performing the at least one motor skill intervention concurrently with the at least one cognitive intervention or the at least one psychological intervention.

In one embodiment the adapting of the therapeutic plan further comprises selecting and implementing therapeutic intervention parameters based on at least one attribute of the individual selected from: age, hobbies, music preferences, spoken language, caregiver involvement preferences, disease characteristics, disease severity, symptoms, educational background, professional background, and individual-defined goals.

In one embodiment the method further comprises administering at least one medicinal agent before, during, after, or a combination thereof, the implementation of at least one of the therapeutic interventions.

In one embodiment the at least one medicinal agent comprises a dopamine precursor and optionally in combination with an adjunct agent.

In one embodiment the dopamine precursor and optionally in combination with an adjunct agent comprises any of the following selected from: levodopa, levodopa/carbidopa, levodopa/benserazide, or combinations thereof.

In one embodiment the therapeutic plan is further adapted to the dopaminergic state of the individual selected from an ON state or an OFF state, wherein the selection, intensity, and scheduling of the therapeutic interventions is optimized based on whether the individual is in the ON state or the OFF state.

In one embodiment the at least one medicinal agent comprises any of the following selected from: dopaminergic agents, dopamine precursors, dopamine agonists, COMT inhibitors, MAO-B inhibitors, peripheral DOPA decarboxylase inhibitors, glutamatergic modulators, anticholinergic agents, antidepressant agents, anxiolytic agents, antipsychotic agents, cognitive enhancing agents, alpha-synuclein targeting agents, LRRK2 inhibitors, adenosine A2A receptor antagonists, GLP-1 receptor agonists, incretin-based therapies, gene therapy agents, viral-vector-mediated gene therapies, neurotrophic factor modulators, sigma-1 receptor modulators, muscarinic receptor modulators, neuroprotective or neuronal-homeostasis-restoring agents, inflammasome modulators, neuro-inflammation modulators, immunomodulators, immunosuppressants, G-protein-coupled receptor modulators, GCase modulators, mucolytic agents, anticonvulsants, beta-blockers, vitamins, cofactor supplements, cannabinoids, symptom-managing adjunct agents, cell-based dopaminergic neuron replacement therapies, and stem-cell-derived dopaminergic neuron replacement therapies, D1/D5 dopamine receptor partial agonists, tavapadon, prasinezumab, BIIB122, DNL151, lixisenatide, exenatide, blarcamesine, NLRP3 inhibitors, GPR6 inverse agonists, and solengepras, or combinations thereof.

In one embodiment the method further comprises administering light therapy before, during, after, or a combination thereof, at least one of the therapeutic interventions.

In one embodiment the method further comprises:
dynamically monitoring the individual's monitored parameters during delivery of the therapeutic plan;
generating a dynamic user profile based on the individual's monitored parameters selected from: the initial profile, Electronic Medical Records (EMR) data, motor psychological attributes, cognitive attributes, pharmacological attributes, feedback of the individual, feedback of a medical practitioner, drug intake parameters, or a combination thereof; and
adapting the therapeutic plan based on the dynamic user profile.

In one embodiment the method further comprises recursively adapting the therapeutic plan based on updates to the dynamic user profile, the performance metrics, and the individual's responses.

In one embodiment the recursive adapting of the therapeutic plan comprises dynamically identifying and modifying intervention parameters to implement optimized therapeutic outcomes towards a predetermined therapeutic goal.

In one embodiment the intervention parameters comprise any of the following selected from: type, timing, dosage, intensity, sequence, duration, frequency, mode of delivery, level of guidance, feedback modality, language adaptation, complexity level, interface configuration, integration with external systems, or a combination thereof, of one or more of the therapeutic interventions.

In one embodiment the recursive adapting is performed using a feedback control algorithm, Bayesian inference, or a combination thereof.

In one embodiment the at least one motor skills intervention is selected from: speech and language therapy, physiotherapy, tremor management, mobility training, freezing and rigidity exercises, tapping training, limb agility exercises, training for freezing of gait, motor function therapy, dance therapy, handwriting training, balance exercises, postural stability training, strength training, stretching exercises, coordination training, metronome training, Tai-chi, physical exercises, walking, dual tasking exercises, and fine motor skills training, or a combination thereof.

In one embodiment the at least one psychological intervention is selected from: guided imagery, psychoeducation, psychotherapy, cognitive behavioral therapy, stress and anxiety management training, mindfulness-based interventions, body scanning training, sleep hygiene, fatigue training, acceptance and commitment therapy (ACT), dialectical behavior therapy (DBT), attention training techniques, psychodynamic therapy, solution-focused brief therapy (SFBT), narrative therapy, pain therapy, addiction therapy, gestalt therapy, behavioral activation therapy, adjustment therapy, grief therapy, motivational therapy, meaning-centered therapy, creative therapy, expressive therapy, logotherapy, telepsychiatry, and teletherapy, or a combination thereof.

In one embodiment the at least one cognitive intervention is selected from: navigation in a maze, navigation in a digital maze, spatial navigation, working memory training, magic-7 training, memory enhancement techniques, executive function training including, problem-solving training, planning training, cognitive flexibility training, inhibition training, attention training, concentration training, processing speed training, sonification exercise, data visualization, geometric puzzles, shape and pattern matching, visual perception tasks, spatial reasoning games, face detection training, drawing game, focus training, reading training, cueing training, metacognition training, memory retrieval training, dual tasking training, or any combination thereof.

In one embodiment each of the therapeutic interventions has a duration ranging between 10 s to 60 mins.

In one embodiment the predetermined condition is selected from: a predetermined number of iterations, a predetermined duration has elapsed, a predetermined number of therapeutic interventions have been completed, predetermined progress measurement, predetermined regression measurement, target threshold score achieved, transition to maintenance therapy, and no further improvement is measured, or a combination thereof.

In one embodiment the targeted interaction outputs are selected from: visual, auditory, tactile, or a combination thereof.

In one embodiment at least one of the therapeutic interventions further comprises: sensory inhibition, sensory substitution, sensory integration, or a combination thereof.

In one embodiment the method further comprises a conditioning stimulus delivered before, during, after, or a combination thereof, the administering of the at least one medicinal agent, at least one of the therapeutic interventions, or a combination thereof.

In one embodiment the Parkinson's disease-related markers comprise any of the following selected from: Unified Parkinson's Disease Rating Scale (UPDRS) score, motor symptoms, tremor amplitude, gait symmetry, or reaction-time variability, cognitive function, autonomic function, fatigue, sleep-related markers, sensory or olfactory function, biochemical markers, molecular markers, dopamine levels, α-synuclein, neuroinflammatory markers, oxidative stress markers, neurotrophic factors, pro-inflammatory cytokines, electrophysiological markers, and imaging-derived measures of dopaminergic function, basal ganglia structure, or functional connectivity, or a combination thereof.

In one embodiment the invention provides a digital therapeutic system for modulating one or more Parkinson's disease-related markers, symptoms, or disease progression in an individual in need thereof, the system comprising:
a data-input module configured to onboard the individual and dynamically monitor and store data related to the individual throughout a therapeutic plan; and
a digital therapy delivery module comprising a processor configured to deliver the therapeutic plan comprising therapeutic interventions selected from: at least one motor skills intervention, at least one psychological intervention, at least one cognitive intervention, or a combination thereof;
wherein the digital therapy delivery module is further configured to convert the therapeutic plan into a plurality of targeted interaction outputs for presentation to the individual and track the individual's responses.

In one embodiment the digital therapy delivery module comprises software executable on a personal electronic device.

In one embodiment the data-input module is configured to receive data from a plurality of data acquisition sources in real-time, wherein the data comprises one or more of: Electronic Medical Records (EMR) data, motion sensor data, position sensor data, environmental sensors data, audio sensors data, location sensors data, audio recording data, speech recognition data, gaze-tracking data, touch interaction data, physiological sensor data, optical sensor, imaging sensors, self-report data, or a combination thereof.

In one embodiment the digital therapy delivery module comprises any of the following selected from: a personal electronic device, a graphical user interface, audio interface, haptic interface, or a combination thereof.

In one embodiment the processor is further configured to:
dynamically monitor any of the following factors of the individual selected from: Electronic Medical Records (EMR) data, motor attributes, psychological attributes, cognitive attributes, pharmacological attributes, and drug intake parameters, or a combination thereof, throughout the implementation of the therapeutic plan;
generate a dynamic user profile, and recursively adapt the therapeutic plan based on the dynamic user profile, performance metrics, and the individual's responses;
or a combination thereof.

In one embodiment the system further comprises at least one additional device operatively coupled to the digital therapy delivery module, selected from: health monitoring systems, medical devices, haptic devices, external speakers, headphones, virtual reality headsets, augmented reality glasses or devices, biofeedback sensors, wearable activity trackers, smartphones, tablets, smartwatches, personal computational devices, smart speakers, voice assistants, motion tracking sensors, virtual assistant systems, internet hubs, gait sensors, eye-tracking devices, voice recording devices, and wearable devices, or combinations thereof.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1 shows a flow chart of the study design of Example 2.

FIGS. 2A-2B show primary endpoints, total MDS-UPDRS results. FIG. 2A shows the mean change from baseline in total MDS-UPDRS score across groups. FIG. 2B shows cumulative distribution of the change from the baseline in total MDS-UPDRS.

FIG. 2C-2E show secondary endpoints: MDS-UPDRS Part II, III and BDI-II results. FIG. 2C shows the mean change from careline in MDS-UPDRS-II score across groups. FIG. 2D shows mean change from baseline in MDS-UPDRS-III score across groups. FIG. 2E shows mean change from baseline in BDI-II score across groups.

FIGS. 3A-3I show results for motor function correlates: clinical, neural, and digital metrics. FIG. 3A shows an illustration of the motor loop. FIG. 3B shows MDS-UPDRS-III improvement correlates with DopApp self-engagement in challenging sensorimotor tasks. DopApp refers to an example of a software application that carries out embodiments of the invention described herein.

FIG. 3C shows enhanced thalamic VL-M1 connectivity. FIGS. 3D-3F show correlation between increased thalamic VL-M1 connectivity and improvement in motor symptoms. FIGS. 3G-3I shows correlation between increased thalamic VL-M1 connectivity and DopApp sensorimotor deprivation engagement.

FIGS. 4A-4I show results for non-motor function correlates: clinical, neural, and digital metrics. FIG. 4A shows an illustration of the limbic loop. FIG. 4B shows MDS-UPDRS-II improvement correlates with DopApp self-engagement in emotion regulation activities. FIG. 4C-4D show enhanced thalamic AV-Cortical connectivity where group-by-time interaction with DopApp is greater than Plaecbo, Post>Pre. FIG. 4E-4G shows correlations between increased thalamic AV connectivity and improvement in depressive symptoms. FIGS. 4H-4I show correlation between increased thalamic AV-mPFC connectivity and self-engagement with DopApp emotion regulation activities.

FIGS. 5A-5B show correlations between changes in Amygdala rsFC and improvement in non-motor symptoms. FIG. 5C shows a graph representing correlations between MDS-UPDRS and BDI-II improvements. FIG. 5D shows correlations between increased Amygdala-Orbitofrontal connectivity and the software application DopApp reward-based tasks. FIG. 5E shows MDS-UPDRS-II improvement correlates with the software application DopApp self-engagement in sensorimotor deprivation tasks. FIG. 5F shows correlations between left Amygdala-Caudate connectivity and the software application DopApp engagement time with emotion regulation. FIG. 5G shows a graph representing MDS-UPDRS-II improvement correlates with the software application DopApp engagement time with emotion regulation. FIG. 5H shows correlation between changes in the left Amygdala connectivity and improvement in depression.

FIG. 6 shows a study flowchart for Example 2.

FIGS. 7A-7C show adherence and satisfaction survey results for the DopApp and placebo interventions.

FIGS. 8A-8B show post-intervention alterations in the DopApp group.

FIGS. 9A-9F show voxel-based Diffusion Tensor Imaging (DTI) for DopApp and placebo intervention groups.

FIGS. 10A-10B show graphical correlations between changes in VLp FA and motor symptom improvement. No significant associations were found in the placebo group.

FIGS. 11A-11I show network topology analysis results. FIGS. 11A-11E show the right PreCG ROI neighborhood network, demonstrating coupling with premotor cortex, basal ganglia and posterior parietal cortex regions. Network nodes were extracted from patients' binary matrices. FIGS. 11F-11I show changes in PreCG post-treatment local efficiency for different edge-weight matrices. Significant group-by-time results were found for average tract lenth and FA edge-weights. FA. fractional anisotropy, MD, mean diffusivity, AAL, Automated Anatomical labeling atlas, PreCG, precentral gyrus, P-value, group-by-time ANOVA model.

FIG. 12 shows a DTI image processing pipeline.

FIGS. 13A-13D show sample screen-shots from the DopApp application for a smart phone. FIG. 13A shows a whack-a-mole game where a user has to hit the object emerging from the hole. FIG. 13B shows a voice-prompted navigation game (‘bird game’) where the user uses voice pitch/loudness/sound to navigate the bird around obstacles. FIGS. 13C-13D show a 2D and 3D digital maze navigation game which utilizes sensory inhibition, sensory substitution, and sensory integration.

For simplicity and clarity of illustration, elements shown in the figures are not necessarily drawn to scale, and the dimensions of some elements may be exaggerated relative to other elements. In addition, reference numerals may be repeated among the figures to indicate corresponding or analogous elements.

### DETAILED DESCRIPTION

The present invention seeks to address these unmet needs by harnessing the potential of digital therapeutics (DTx), which are software-based products designed to provide tailored care to patients. The invention is a system and method for treating PD, which is comprised of an integrated set of non-pharmacological interventions, which can be implemented independently as a standalone therapy or in a hybrid mode in combination with pharmacological interventions, where it is synchronized with the drug and enhances its efficacy. The method is delivered through a digital platform, such as a smartphone application.

The method comprises the integration any number of complimentary principal digital modes of action, which are designed to enhance the following peripheral and neural processes, into a single treatment protocol: (1) improving motor control, (2) reducing systemic & neuro inflammation, (3) enhancing brain plasticity, (4) rebalancing emotional & cognitive networks, and (5) triggering the reward system. These modes will be elaborated upon in the following paragraphs.

In the context of treating PD, which is characterized by a wide range of symptoms that can manifest differently in each patient, the approach underlying the present invention implements a holistic methodology that is both comprehensive and adaptable to the individual needs of patients. Given the variability in symptom presentation across patients, this approach ensures that treatment protocols are not static but are instead dynamic, evolving in response to patient- or disease-specific parameters. These parameters may include but are not limited to the stage of the disease, variations in symptom severity, progression rate of the disease, and individual biological markers. As a result, the treatment method continuously adapts to optimize outcomes, ensuring that it remains aligned with the unique manifestation of the disease in each patient.

While the ideal treatment protocol involves the incorporation of all five modes of action, in certain embodiments of this invention, the protocols of treatment include only a subset (one or more than one) of these modes, and in other treatment protocols, certain modes are emphasized more than others at different points in the therapeutic process.

Some of the digital interventions integrated in the protocol are designed primarily to deliver one of the principal modes of action described above and address specific aspects of the disease. However, in some cases, a single intervention may span more than one mode of action.

Some of the interventions-primarily ones directly aimed at enhancement of brain plasticity, but other interventions as well—are designed in a manner that leverages the “sensory principles”, which are: sensory inhibition, sensory substitution, sensory integration, or a combination thereof. Sensory inhibition (or masking) involves the partial or complete suppression of one sensory input, such as reducing visual or auditory stimuli, to alter the individual's sensory experience. For example, covering one or both eyes or lowering the volume to suppress the visual or auditory input, respectively. Sensory substitution replaces one sensory modality with another, such as replacing visual stimuli with auditory ones, or vice versa. Sensory integration involves the combination of multiple sensory inputs, such as synchronizing auditory, visual, and tactile cues to enhance the sensory experience. For instance, the sound of waves breaking could be paired with a visual of the ocean and a tactile vibration, creating a more immersive, multi-sensory experience. As stated above, many of the interventions utilize the Sensory Principles, and even interventions that do not directly employ these principles are interwoven within the same treatment protocol with those targeting brain plasticity, which typically utilize them. The enhancement of brain connectivity amplifies other therapeutic outcomes targeted by the various interventions. This integration strengthens the effectiveness of interventions aimed at improving cognitive, emotional, motor, immune, reward, or stress-related functions, and ensuring that these outcomes are more long-lasting. All of this reflects the broader understanding that all learning and adaptation are fundamentally driven by brain plasticity.

As used herein, the term “modality” refers to therapeutic interventions designed to target specific functional domains relevant to the treatment of Parkinson's disease. Modalities include, but are not limited to: motor skills interventions (e.g., exercises to improve gait, dexterity, or coordination), cognitive interventions (e.g., tasks to enhance memory, attention, or executive function), and psychological interventions (e.g., techniques to support mood regulation, emotional resilience, or stress reduction).

As used herein, “multi-modality” refers to the integration of two or more such therapeutic modalities within a unified treatment plan. By combining interventions across domains, the system enables synergistic effects. This integrative approach supports optimization of therapeutic outcomes, allowing the system to dynamically adjust treatment based on evolving user needs and performance data. Furthermore, when therapeutic interventions are delivered in combination with the administration of medicinal agents, this introduces an additional dimension, or modality, into the integrated treatment plan, enhancing synergism.

### Methods of the Invention

In one embodiment the invention provides a method for modulating one or more Parkinson's disease-related markers, symptoms, or disease progression in an individual in need thereof, the method comprising:
extracting a plurality of attributes from the individual from a plurality of data acquisition sources to generate an initial profile for the individual;
generating and/or personalizing a therapeutic plan based on the initial profile, wherein the therapeutic plan comprises therapeutic interventions selected from: at least one motor skills intervention, at least one psychological intervention, at least one cognitive intervention, or a combination thereof;
converting the therapeutic plan into a plurality of targeted interaction outputs for presentation to the individual; and
adaptively delivering the therapeutic plan, tracking the individual's responses and associated performance metrics, and continuing until a predetermined condition is met.

In one embodiment the invention provides a method for modulating one or more Parkinson's disease-related markers, symptoms, or disease progression in an individual in need thereof, the method comprising:
extracting a plurality of attributes from the individual from a plurality of data acquisition sources to generate an initial profile for the individual;
generating, personalizing, or a combination thereof, a therapeutic plan based on the initial profile, wherein the therapeutic plan comprises therapeutic interventions selected from: at least one motor skills intervention, at least one psychological intervention, at least one cognitive intervention, or a combination thereof;
converting the therapeutic plan into a plurality of targeted interaction outputs for presentation to the individual; and
adaptively delivering the therapeutic plan, tracking the individual's responses and associated performance metrics, and continuing until a predetermined condition is met.

In one embodiment the invention provides a method for modulating one or more Parkinson's disease-related markers, symptoms, or disease progression in an individual in need thereof, the method comprising:
extracting a plurality of attributes from the individual from a plurality of data acquisition sources to generate an initial profile for the individual;
generating, personalizing, or a combination thereof, a therapeutic plan based on the initial profile, wherein the therapeutic plan comprises therapeutic interventions selected from: at least one motor skills intervention, at least one psychological intervention, at least one cognitive intervention, or a combination thereof;
converting the therapeutic plan into a plurality of targeted interaction outputs for presentation to the individual; and
adaptively delivering the therapeutic plan, tracking the individual's responses and associated performance metrics.

In one embodiment the invention provides a method for modulating one or more Parkinson's disease-related markers, symptoms, or disease progression in an individual in need thereof, the method comprising:
determining a therapeutic plan to be delivered to the individual, wherein the therapeutic plan comprises therapeutic interventions selected from: at least one motor skills intervention, at least one psychological intervention, at least one cognitive intervention, or a combination thereof;
converting the therapeutic plan into a plurality of targeted interaction outputs for presentation to the individual; and
adaptively delivering the therapeutic plan, tracking the individual's responses and associated performance metrics, and continuing until a predetermined condition is met.

As used herein, the term “modulating” refers to changing, adjusting, modifying, disease modification, altering, influencing, or affecting the level, magnitude, pattern, rate, variability, expression, manifestation, severity, or progression of a parameter, condition, symptom, or marker. Modulation may include increasing, decreasing, enhancing, improving, amplifying, reducing, attenuating, suppressing, stabilizing, normalizing, treating, or delaying the onset or progression of such parameter. Modulation may occur in any direction and may represent a partial, complete, transient, sustained, direct, or indirect effect. The modulation may be assessed relative to a baseline, control condition, population norm, or expected natural disease trajectory.

As used herein, a “Parkinson's disease-related marker” refers to any clinical, behavioral, physiological, biochemical, molecular, electrophysiological, or imaging-derived measure that is indicative of Parkinson's disease presence, severity, progression, symptom expression, treatment response, or functional status. Such markers include, but are not limited to: standardized clinical rating scales (e.g., Unified Parkinson's Disease Rating Scale (UPDRS) score, Beck Depression Inventory (BDI) scale, Patient Global Impression of Change (PGIC) scale, Clinical Global Impression-Improvement (CGI-I) scale), motor performance measures (e.g., tremor amplitude, bradykinesia metrics, gait symmetry or variability), reaction-time or cognitive performance measures, autonomic function assessments, fatigue or sleep-related measurements, sensory or olfactory function measures, biochemical or molecular biomarkers (e.g., dopamine levels, α-synuclein, neuroinflammatory or oxidative stress markers, neurotrophic factors, pro-inflammatory cytokines), electrophysiological readouts (e.g., local field potential activity or neural firing patterns), and neuroimaging-derived measures (e.g., dopaminergic function, basal ganglia structure or connectivity). A Parkinson's disease marker may be quantitative or qualitative, direct or indirect, and may reflect either a state value or a change over time. Any combination of such markers may be used.

As used herein, and in one embodiment, “converting the therapeutic plan into targeted interaction outputs” refers to the transformation of selected therapeutic interventions into user-perceivable stimuli and interactive elements that can be delivered through a digital interface. This process involves mapping each intervention type to appropriate output formats based on its functional goals, the capabilities of the user interface, and the individual's preferences or accessibility needs. For example, motor skills interventions may be rendered as animated exercise demonstrations, touch-sensitive targets for coordination tasks, audio prompts for vocal exercises, or haptic feedback for rhythm training. Psychological interventions may be presented as video content for guided imagery, audio narration for mindfulness, or interactive questionnaires for cognitive behavioral therapy. Cognitive interventions may take the form of visual mazes with navigational controls, memory matching games, or audio-visual puzzle interfaces. The conversion process ensures that each therapeutic element is delivered in a format that is engaging, accessible, and aligned with the intended therapeutic outcome. In certain embodiments, determining the therapeutic plan may encompass any suitable mode of obtaining or defining the therapeutic plan for the individual. For example, the therapeutic plan may be acquired from an existing clinical protocol, guideline, standardized care pathway, or previously established therapy regimen. In other embodiments, the therapeutic plan may be generated de novo based on patient-specific characteristics, assessments, goals, or clinician input. In one embodiments, the therapeutic plan may be adapted or modified from a pre-existing plan, such as by revising one or more therapeutic elements, dosing schedules, behavioral interventions, or support activities in accordance with patient needs or observed responses. Any combination of acquiring, generating, and adapting a therapeutic plan is also contemplated, and the plan may continue to evolve dynamically over time in response to ongoing monitoring or feedback.

As used herein, “in need thereof” refers to any individual who may benefit from, require, or be a candidate for the disclosed therapeutic interventions, regardless of the stage, severity, or formal diagnosis of a disease e.g., Parkinson's disease.

Although the therapeutic interventions of the invention provide methods of treating, preventing, or alleviating symptoms in an individual affected by various diseases, the methods can also be utilized by someone who is otherwise not affected by these diseases. For example, a person who aims at increasing his/her general wellbeing by improving neuro-logical function. Furthermore, any of the digital therapy interventions, and their corresponding aims (e.g., increasing neuro-connectivity, improving immune function, etc.) can be understood as used to either treat, prevent, alleviate symptoms, or a combination thereof. Therefore “treat, prevent, alleviate symptoms” can be understood as doing each of them separately, or in combination, in various embodiments. Since the methods of the present invention are effective in any of those areas, their capability to treat/prevent/alleviate symptoms, are often considered together.

As used in this description of the present invention, the terms “drug” or “drug therapy” shall encompass treatment with any medication, whether approved or not by the relevant regulatory authorities (such as the FDA).

As used herein, the individual using or undergoing the therapeutic interventions is referred to in a number of ways. For example, a “user” can also be referred to, and understood interchangeably, as a “patient”, “individual”, “subject”, “participant”, “recipient”.

As used herein, “therapeutic plan” refers to a structured set of therapeutic interventions, strategies, or activities. The therapeutic plan may be personalized based on individual attributes, clinical data, behavioral responses, or performance metrics, and may include motor skills interventions, psychological interventions, cognitive interventions, or combinations thereof. It may be delivered digitally, adaptively modified over time, and presented through targeted interaction outputs.

As used herein “psychological” interventions are generally directed towards interventions that affect psychological and/or behavioral aspects of an individual. As such, psychological interventions are generally related to behavioral, social and psychological tasks, as will be described. As used herein “physical” interventions are generally directed towards interventions that improve motor functions in an individual

As used herein, “adaptively delivering” refers to the continuous and responsive administration of the therapeutic plan to the individual, wherein delivery occurs in a manner that dynamically adjusted over time based on the individual's responses, performance metrics, and evolving therapeutic needs. This includes ongoing presentation of targeted interaction outputs as and when needed throughout the course of the therapeutic plan, with modifications to timing, content, intensity, or modality to optimize therapeutic outcomes and align with predetermined conditions.

Examples of adaptive delivery include: Increasing difficulty of a digital maze by 20% when the user achieves >90% accuracy for 3 consecutive sessions; Reducing session frequency from daily to every-other-day when the user reports fatigue; Switching from visual-based to audio-based navigation cues when visual performance declines; Advancing from simple tapping exercises to dual-task tapping+counting when motor performance improves by >15%.

In one embodiment the method further comprises obtaining a plurality of attributes associated with the individual from a plurality of data acquisition sources to generate an initial profile for the individual, wherein the determining of the therapeutic plan further comprises adapting the therapeutic plan based on the initial profile. In various embodiments machine learning is employed to assist in any of the following selected from: generating the initial profile, generating the dynamic user profile, adapting the therapeutic plan, adapting the therapeutic intervention, recursive adaptations, Bayesian inference, feedback mechanisms, performance tracking, data analysis, or combinations thereof. In one embodiment the at least one therapeutic intervention comprises real-time adaptive feedback based on sensor input during performance of the intervention.

In one embodiment the extracting of the plurality of attributes comprises synchronizing multimodal data streams from the plurality of data acquisition sources into a time-aligned dataset, the synchronizing comprising associating each data stream with a unified temporal reference such that data samples, features, or events from the plurality of data acquisition sources correspond to common timestamps or time intervals, thereby enabling identification of temporal relationships and co-occurring patterns across modalities, and wherein the multimodal data streams comprise two or more of: motion data, speech data, gaze-tracking data, physiological signals, touch interaction data, accelerometer data, gyroscope data, pressure sensor data, keyboard and/or touchscreen interaction patterns, facial expression data, autonomic nervous system signals, or response-timing data. As used herein, “multimodal data streams” (as related to “data stream modalities”) refer to multiple types of data collected from different sources, such as motion, speech, touch, and physiological signals, that together provide a comprehensive view of user behavior and state.

In one embodiment the synchronizing comprises timestamping each data point from each data stream modality with a common time reference to enable temporal correlation analysis across data stream modalities and detection of cross-domain patterns indicative of Parkinson's disease progression or therapeutic response. In one embodiment the methods further comprises performing data quality assessment on the multimodal data streams, wherein data quality assessment comprises one or more of: detecting missing data, identifying sensor malfunction, filtering noise, detecting outliers, or validating data integrity. In one embodiment the method further comprises applying data imputation techniques to estimate missing data points based on historical patterns, data from other data stream modalities, or population-level models. In one embodiment the method further comprises integrating data from motor attributes, psychological attributes, cognitive attributes, and pharmacological attributes to generate a unified therapeutic response model that characterizes the individual's multidimensional response to the therapeutic plan. As used herein a “unified therapeutic response model” refers to a comprehensive framework that combines diverse data sources to represent how an individual responds to a therapeutic plan over time. As used herein the term “multidimensional response” refers to the individual's combined behavioral, physiological, cognitive, and emotional changes resulting from therapeutic interventions and medication. In one embodiment the unified therapeutic response model comprises quantified relationships between: motor intervention performance and motor symptom changes; cognitive intervention performance and cognitive function changes; psychological intervention engagement and psychological state changes; pharmacological state and performance across all intervention types; and cross-domain effects wherein interventions in one domain affect outcomes in other domains. As used herein “domain” refers to a distinct functional area targeted by therapeutic interventions, such as motor function, cognitive ability, psychological state, or pharmacological status. In one embodiment the unified therapeutic response model enables holistic assessment of the individual's Parkinson's disease state by capturing interactions and compensatory mechanisms across motor, cognitive, psychological, and pharmacological domains that would not be apparent from assessment of individual domains in isolation.

Examples of data acquisition sources includes, but are not limited to: electronic medical record (EMR) data, clinical practice guidelines, evidence-based protocols, patient input, expert consultation including medical professionals, allied health professionals, and user experience specialists, and machine learning analysis of aggregated patient outcome data to identify and optimize effective therapeutic intervention combinations. Electronic Medical Records (EMR) refers to digital medical records maintained by a healthcare provider for an individual patient. Comprised within that, the EMR data can include any of the following: patient demographics, medical history and prior diagnoses, current and past medications and prescriptions, documented allergies and contraindications, vital signs and other routine clinical measurements, laboratory test results, imaging reports (e.g., X-ray, CT, MRI), clinician notes, treatment plans and care instructions, and records of visits, appointments, and clinical encounters.

In one embodiment two or more of the therapeutic interventions are carried out simultaneously. For example, one motor skill intervention and cognitive intervention are carried out simultaneously, or two motor skills are carried out simultaneously. In one embodiment the wherein the therapeutic intervention is selected from at least one motor skills intervention, at least one psychological intervention, and at least one cognitive intervention.

In one embodiment the therapeutic plan comprises performing the at least one motor skill intervention concurrently with the at least one cognitive intervention or the psychological intervention. In one embodiment, the therapeutic plan incorporates dual tasking, wherein the individual is engaged in two or more distinct therapeutic interventions simultaneously. Dual tasking may include, for example, performing a motor skill activity while concurrently engaging in a cognitive or psychological task.

The digital platform may support concurrent or dual-task therapeutic interventions in which two or more modalities are executed simultaneously. For example, a cognitive maze-navigation exercise may occur concurrently with a vocal pitch-control task or guided breathing sequence, thereby engaging motor, cognitive, and psychological domains in a unified adaptive session. The system may dynamically balance the load between the tasks based on real-time monitoring of the user's performance and fatigue levels.

In one embodiment the therapeutic plan further comprises incorporating at least one personal preference of the individual selected from: age-appropriate content, hobby-related themes, preferred music genres, spoken language, caregiver involvement level, educational background, professional background, or a combination thereof, wherein the therapeutic plan is adapted according to the at least one personal preference. Any of these personal preferences can be input into the systems of the invention for the purpose of updating an individual's profile, updating the therapeutic plan, or a therapeutic intervention itself.

In one embodiment the therapeutic plan further comprises incorporating at least one personal preference of the individual selected from: age-appropriate content, hobby-related themes, preferred music genres, spoken language, caregiver involvement level, educational background, professional background, patient-defined goals, or a combination thereof, wherein the therapeutic plan is adapted according to the at least one personal preference.

Examples of patient-defined goals include, but are not limited to: improving mobility, enhancing cognitive function, reducing tremor, improving speech intelligibility, increasing daily activity levels, improving sleep quality, improving social activity levels, managing stress and anxiety, and managing mood, or a combination thereof.

In one embodiment the adapting further comprises selecting and implementing therapeutic intervention parameters based on at least one attribute of the individual selected from: age, hobbies, music preferences, spoken language, caregiver involvement preferences, disease characteristics, disease severity, symptoms, educational background, professional background, and individual-defined goals.

Combination with Medicinal Agents

As explained above, digital interventions of the types explored herein can be used on a stand-alone basis or in combination with at least one medicinal agent. Generally, an adjunctive medication is a supplementary therapeutic agent used in conjunction with primary treatment to optimize efficacy or address specific aspects of a medical condition, often enhancing overall therapeutic outcomes. As will become clear, any therapeutic agent that achieves this goal in a combination with the digital therapy of the invention is considered within the scope of the invention.

As used herein the term “combined” (or “combined method”) describes any intervention that includes at least one digital therapy intervention and at least one non-digital therapy intervention e.g., a medicinal agent.

In various embodiments, and as used herein, the terms “drug”, “medication”, “medicinal agent”, “pharmaceutical”, “medicine” or the likes are to be understood interchangeably. Namely, these refer to any substance used to diagnose, prevent, treat, or alleviate symptoms of a medical condition. They can also refer to substances that are generally aimed at maintaining or improving an individual's well-being without specifically targeting a disease. Thus, in various embodiments, the ‘medicinal agent’ can refer to a nutritional supplement, as will be detailed. As used herein, and in various embodiments, the “treatment” of any disease includes any intervention that alleviates symptoms or causes at least one marker of that disease to improve. Therefore, “treatment”, as understood herein, also refers to delayed onset or prevention of a disease, in various embodiments.

In one embodiment the method further comprises administering at least one medicinal agent before, during, after, or a combination thereof, the implementation of at least one of the therapeutic interventions. In one embodiment the method further comprises administering the at least one medicinal agent at a time selected from: prior to, concurrently with, subsequent to, or any combination thereof, the delivery of at least one of the therapeutic interventions.

In one embodiment the at least one medicinal agent comprises a dopamine precursor and optionally in combination with an adjunct agent.

As used herein, “dopamine precursor” refers to any pharmacological agent or compound that modulates the dopaminergic state of the individual. For example the dopamine precursor is configured to modulates dopaminergic state of the individual by enhancing, supporting, or facilitating the synthesis, availability, or activity of dopamine in the individual. Such agents may induce an ON state or contribute to a transition between ON and OFF states, depending on timing, dosage, and individual response. As used herein, “adjunct agent” refers to any compound co-administered with a primary therapeutic agent (e.g., dopamine precursor) to enhance or modulate its effect.

In one embodiment adapting the therapeutic plan to the dopaminergic state involves both selecting appropriate intervention types and adjusting intervention parameters based on whether the individual is in the ON state or OFF state. For example, in the ON State (when medication effect is present) emphasized interventions may include, but are not limited to: motor interventions requiring fine motor control (tapping tests, handwriting), balance and postural stability exercises requiring good motor function, dual-task interventions combining motor and cognitive demands, more challenging therapeutic intervention difficulty levels, faster-paced sensorimotor games. On the other hand, for example, in the OFF State (when medication effect is reduced/absent), emphasized interventions may include, but are not limited to: cognitive interventions that do not heavily rely on motor function, psychological interventions (guided imagery, mindfulness), emotion regulation content, less motor-demanding activities, simpler cognitive tasks that reduce frustration.

For Parkinson's therapeutic interventions, intensity is dynamically modified based on the individual's motor state. During the ON state, when medication is active and motor function is more stable, the system increases challenge by applying higher difficulty levels, faster tempos, smaller touch targets, and more complex interaction patterns. In contrast, during the OFF state, when motor symptoms are more pronounced, the intervention adapts by reducing difficulty, slowing tempos, enlarging touch targets, and simplifying task patterns to maintain accessibility and engagement.

Scheduling adaptations are applied to align therapeutic interventions with the individual's medication cycle and motor state. Motor-intensive interventions are scheduled during typical ON periods, such as 60 to 90 minutes after a levodopa dose, when motor function is more stable and responsive. Cognitive and psychological interventions are timed during OFF periods or independently of medication timing, as they are less reliant on optimal motor performance. To prevent frustration or reduced efficacy, demanding interventions are avoided during typical wearing-off periods, such as 3 to 4 hours post-dose, when symptoms may re-emerge.

In one embodiment the system determines the individual's current dopaminergic state using multiple complementary methods. First, through user self-report, the individual can indicate their perceived ON or OFF state via a simple interface toggle. Second, the system applies time-based prediction, estimating the expected motor state based on the time elapsed since the last logged medication dose and known pharmacokinetic profiles, for example, levodopa typically peaks at 60-90 minutes and begins wearing off by 3-4 hours. Third, performance-based inference is used, where the system analyzes real-time metrics such as tremor amplitude (from accelerometer data), tapping speed, and movement smoothness, comparing these to the user's historical ON and OFF performance profiles to infer the likely state. Finally, a hybrid approach may be employed, combining self-report, time-based modeling, and performance analytics to improve accuracy and responsiveness in state detection.

In one embodiment the dopamine precursor and optionally in combination with an adjunct agent comprises any of the following selected from: levodopa, levodopa/carbidopa, levodopa/benserazide, or combinations thereof.

In one embodiment the therapeutic plan is further adapted to the dopaminergic state of the individual selected from an ON state or an OFF state, wherein the selection, intensity, and scheduling of the therapeutic interventions is optimized based on whether the individual is in the ON state or the OFF state.

In one embodiment the dopaminergic agent comprises any of the following selected from levodopa, pramipexole, ropinirole, rotigotine, bromocriptine, or combinations thereof, and wherein the adjunct agent comprises any of the following selected from: carbidopa, benserazide, or combinations thereof.

In one embodiment the at least one medicinal agent comprises a dopaminergic agent potentially inducing ON and OFF states in Parkinson's disease, optionally in combination with an adjunct agent. In one embodiment the dopaminergic agent is levodopa. In one embodiment the adjunct agent comprises: carbidopa, benserazide, or a combination thereof. In one embodiment the at least one medicinal agent comprises a dopaminergic agent configured to induce ON and OFF states in Parkinson's disease, optionally in combination with an adjunct agent. In one embodiment the dopaminergic agent is levodopa. In one embodiment the adjunct agent comprises: carbidopa, benserazide, or a combination thereof.

In one embodiment the at least one medicinal agent comprises any of the following selected from: dopaminergic agents, dopamine precursors, dopamine agonists, COMT inhibitors, MAO-B inhibitors, peripheral DOPA decarboxylase inhibitors, glutamatergic modulators, anticholinergic agents, antidepressant agents, anxiolytic agents, antipsychotic agents, cognitive enhancing agents, alpha-synuclein targeting agents, LRRK2 inhibitors, adenosine A2A receptor antagonists, GLP-1 receptor agonists, incretin-based therapies, gene therapy agents, viral-vector-mediated gene therapies, neurotrophic factor modulators, sigma-1 receptor modulators, muscarinic receptor modulators, neuroprotective or neuronal-homeostasis-restoring agents, inflammasome modulators, neuro-inflammation modulators, immunomodulators, immunosuppressants, G-protein-coupled receptor modulators, GCase modulators, mucolytic agents, anticonvulsants, beta-blockers, vitamins, cofactor supplements, cannabinoids, symptom-managing adjunct agents, cell-based dopaminergic neuron replacement therapies, and stem-cell-derived dopaminergic neuron replacement therapies, or combinations thereof. In one embodiment the at least one medicinal agent comprises any of the following selected from: D1/D5 dopamine receptor partial agonists, tavapadon, prasinezumab, BIIB122, DNL151, lixisenatide, exenatide, blarcamesine, NLRP3 inhibitors, GPR6 inverse agonists, and solengepras, or combinations thereof.

Examples of symptom-managing adjunct agents includes, but are not limited to: analgesics, sleep aids, blood pressure regulators.

In one embodiment the method further comprises administering light therapy before, during, after, or a combination thereof, at least one of the therapeutic interventions. In one embodiment the light therapy is delivered by a light therapy system. In other embodiments the light therapy is delivered by a personal electronic device. In one embodiment the light therapy is delivered by the same system that delivers the therapeutic interventions.

Light therapy may be administered before, during, or after the implementation of the therapeutic interventions. When applied prior to digital sessions, the light therapy may act as a priming stimulus, enhancing alertness and dopaminergic activation. When delivered concurrently, it may augment sensory engagement, and when applied post-session, it may promote neuroplastic consolidation. In some embodiments, the timing and wavelength parameters of the light therapy are dynamically synchronized with the adaptive state of the user.

In one embodiments, the light therapy comprises delivery of visible light within a therapeutic wavelength range selected to improve motor-related neurological function. In one embodiment the light therapy comprises delivering a wavelength of between approximately 520 nm and 570 nm. In one embodiment the wavelength ranges between 490 nm and 570 nm. The light therapy is configured to deliver at an above-ambient intensity sufficient to induce a therapeutic effect. In some embodiments, the light source may be configured to substantially limit or prevent ocular exposure to wavelengths outside of the therapeutic range, such as by incorporating one or more optical filters that block, or attenuate wavelengths known to exacerbate symptoms associated with the motor-related neurological condition. In these embodiments, the intensity of non-therapeutic or symptom-exacerbating wavelengths may be reduced to ambient or below-ambient levels, while the therapeutic wavelengths are preferentially transmitted. In one embodiment the light therapy system may include a control element that allows selective switching between modes that emit therapeutic wavelengths and modes that emit symptom-exacerbating wavelengths, the latter being useful for diagnostic evaluation, symptom provocation testing, progression monitoring, or therapy titration.

In one embodiment the method further comprises:
dynamically monitoring the individual's monitored parameters during delivery of the therapeutic plan;
generating a dynamic user profile based on the individual's monitored parameters selected from: the initial profile, motor attributes, psychological attributes, cognitive attributes, pharmacological attributes, feedback of the individual, feedback of a medical practitioner, drug intake parameters, or a combination thereof; and
adapting the therapeutic plan based on the dynamic user profile.

The dynamic monitoring, generating of a dynamic user profile and adapting the therapeutic plan applies to both ‘stand-alone’ intervention therapies and combination therapies. In the stand-alone context, the therapeutic plan may be delivered without any medicinal agents, relying solely on motor, psychological, and cognitive interventions. In the combination therapy context, the therapeutic plan may be delivered alongside at least one medicinal agents, such as dopamine precursors (e.g., levodopa), with the monitored parameters including pharmacological attributes and drug intake data. The dynamic user profile and adaptive adjustments to the therapeutic plan are applicable in either case, enabling personalized and responsive therapy regardless of whether medicinal agents are administered.

As used herein, “dynamic user profile” refers to a continuously updated representation of an individual's profile, attributes, behaviors, responses, and performance metrics collected during the delivery of a therapeutic plan. The dynamic user profile can include a historical archive of any of these parameters and attributes.

In one embodiment the method further comprises recursively adapting the therapeutic plan based on updates to the dynamic user profile, the performance metrics, and the individual's responses.

As used “recursively adapting” refers to an iterative and ongoing process of modifying the therapeutic plan based on updated information, including changes to the dynamic user profile, performance metrics, and individual responses, and the likes.

In one embodiment the recursive adapting of the therapeutic plan comprises dynamically identifying and modifying intervention parameters to implement optimized therapeutic outcomes towards a predetermined therapeutic goal. In embodiment the predetermined therapeutic goal is the same as the predetermined condition. Examples of predetermined therapeutic goals include, but are not limited to: improving executive function, reducing depressive symptoms, enhancing emotional regulation, increasing gait speed, reducing tremor amplitude, achieving independence in motor tasks, stabilizing medication response, maintaining therapeutic plasma levels, minimizing motor fluctuations, achieving consistent therapy adherence, improving sleep quality, enhancing social interaction, and achieving a target score on a standardized assessment, etc.

In one embodiment the intervention parameters comprise any of the following selected from: type, timing, dosage, intensity, sequence, duration, frequency, mode of delivery, level of guidance, feedback modality, language adaptation, complexity level, interface configuration, integration with external systems, or a combination thereof, of one or more of the therapeutic interventions.

In one embodiment, the intervention parameters comprise changes to the dosage regimen. For example, if the individual undergoes a change in dosage, the therapeutic plan is adapted to reflect and accommodate the updated regimen.

In one embodiment the recursive adapting is performed using a feedback control algorithm, Bayesian inference, or a combination thereof.

In certain embodiments, the recursive adaptation process is implemented using one or more feedback control algorithms that continuously adjust intervention parameters based on deviation from desired performance metrics. For example, a proportional-integral-derivative (PID) control loop or Bayesian inference model may be employed to probabilistically optimize task difficulty, duration, or modality based on the individual's ongoing performance, engagement levels, and sensor-derived data streams. In some embodiments, the adaptive algorithm operates across multimodal data inputs including motion sensors, accelerometers, gyroscopes, microphones, speech analysis, heart rate, or facial expression detection to dynamically refine the therapeutic plan in real time.

In one embodiment the at least one motor skills intervention is selected from: speech and language therapy, physiotherapy, tremor management, mobility training, freezing and rigidity exercises, tapping training, limb agility exercises, training for freezing of gait, motor function therapy, dance therapy, handwriting training, balance exercises, postural stability training, strength training, stretching exercises, coordination training, metronome training, Tai-chi, physical exercises, walking, dual tasking exercises, and fine motor skills training, or a combination thereof.

Examples of speech and language therapy include, but are not limited to: voice therapy, speech therapy, swallow therapy, breathing training, saliva and drooling therapy, chewing therapy, volume-duration-pitch, visual-guided voice training, training facial expression training, dual task training, or a combination thereof.

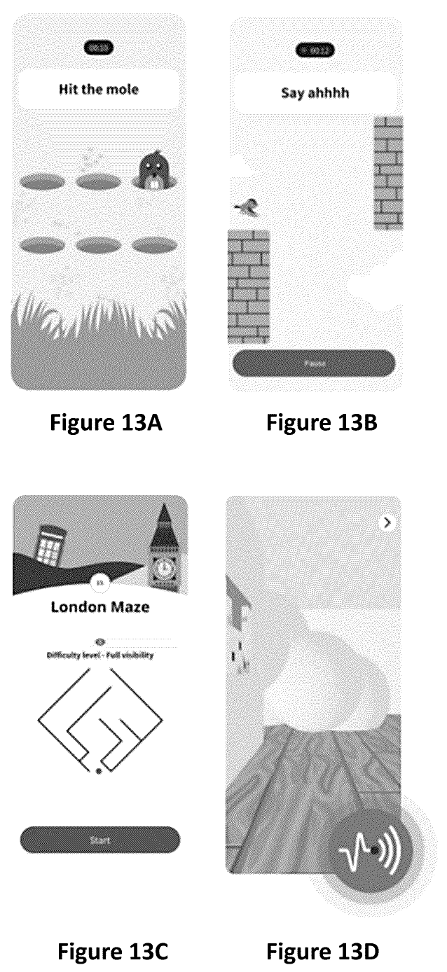

In one example, a voice-controlled navigation game is used in which a character remains fixed in the horizontal position while a continuously scrolling scene presents obstacles. An individual's vocal output, such as pitch or loudness, is used to control the character's vertical movement, allowing the user to raise or lower the character to avoid oncoming obstacles. This voice-prompted, gamified interaction supports therapeutic goals such as speech production, breath control, and general vocal training. For instance, as illustrated in FIG. 13B, a “bird game” is shown in which the user is prompted to say a specific phrase, causing a bird avatar to move up or down to navigate between obstacles such as walls.

In one embodiment the at least one psychological intervention is selected from: guided imagery, psychoeducation, psychotherapy, cognitive behavioral therapy, stress and anxiety management training, mindfulness-based interventions, body scanning training, sleep hygiene, fatigue training, acceptance and commitment therapy (ACT), dialectical behavior therapy (DBT), attention training techniques, psychodynamic therapy, solution-focused brief therapy (SFBT), narrative therapy, pain therapy, addiction therapy, gestalt therapy, behavioral activation therapy, adjustment therapy, grief therapy, motivational therapy, meaning-centered therapy, creative therapy, expressive therapy, logotherapy, telepsychiatry, and teletherapy, or a combination thereof

In one embodiment the at least one cognitive intervention is selected from: navigation in a maze, navigation in a digital maze, spatial navigation, working memory training, magic-7 training, memory enhancement techniques, executive function training including, problem-solving training, planning training, cognitive flexibility training, inhibition training, attention training, concentration training, processing speed training, sonification exercise, data visualization, geometric puzzles, shape and pattern matching, visual perception tasks, spatial reasoning games, face detection training, drawing game, focus training, reading training, cueing training, metacognition training, memory retrieval training, dual tasking training, or any combination thereof.

An example of a digital maze is shown in FIGS. 13C-13B where a user is first presented with a 2D image of the digital maze to be navigated (at a set difficulty). The user then enters a 3D representation of the 2D maze and navigates to an exit point. At first, the user completes the maze using visual prompting i.e., seeing his way through the maze and navigating by swiping (or other). In other embodiments, the digital maze employs any of: sensory inhibition, sensory substitution, sensory integration, or a combination thereof. FIG. 13D shows a ‘cloud’ hindering visibility when navigating the digital maze i.e., an example of sensory inhibition where visibility is impaired. In other embodiments the digital maze may include sound prompts as replacement for visual (or other) prompts, signifying sensory substitution of one sensory modality output with another sensory modality output (at least partially). In this example, as the user progresses through levels of difficulty two sensory modality outputs may be combined together for navigation. For example, the user is not presented with a visual representation of the maze, but must navigate using audio and tactile prompts together e.g., when nearing a wall a sound gets louder and when hitting the wall a vibration prompt is used. This way, a plurality of sensory modality outputs are used separately, and in combinations, to achieve integrative modality use.

The digital mazes of the invention comprise allocentric elements, egocentric elements, or a combination thereof, in various embodiments. In an “allocentric” frame of reference, directions and locations are defined relative to external landmarks or a global coordinate system, independent of the observer's position or orientation. In the context of a digital maze, an allocentric maze is designed such that the layout and structure remain consistent regardless of the perspective of the observer. The maze's walls, paths, and landmarks would be fixed in relation to each other, and the observer navigates by referencing these fixed features. For example, in a digital allocentric maze, a path might be described as “turn right at the T-junction” or “go straight until you reach the blue door. In contrast, an “egocentric” frame of reference defines directions and locations relative to the observer's own position and orientation. In a digital egocentric maze, the layout and structure of the maze would appear differently depending on the observer's perspective. For example, if the observer turns left, the maze's layout would shift accordingly, and what was previously on the left might now be on the right. Directions within an egocentric maze would be described in relation to the observer's current position and orientation. For instance, a direction might be given as “turn left” or “move forward two steps”. Both aspects can be incorporated into the digital mazes of the invention.

A digital maze is one example of a therapeutic intervention. A digital maze intervention comprises using visual, audio, and tactile sensory modality outputs. In various embodiments the digital maze employs: sensory inhibition, sensory substitution, sensory integration, or a combination thereof.

Methods and processes that carry out digital mazes are referred to as a “computer-implemented” method. It refers to any method or process that uses a device to carry out a computation. For example, the computer-implemented method uses at least one electronic device, which can compute, perform tasks, manipulate data, process digital information, execute algorithms, support software. The computer-implemented method encompasses any functionality that relies on the computational power, storage capabilities, and processing abilities of electronics devices, as described herein. The computer-implemented method can execute instructions or algorithms, by processors or computing devices. Instructions may be stored in a computer-readable memory, such as random-access memory (RAM), read-only memory (ROM), or storage devices such as hard drives or solid-state drives. In various embodiments, the methods of the invention provide such computer-implemented methods, by means of a personal electronic to carry out various tasks, including a digital maze that an individual navigates.

In one embodiment the invention provides a computer-implemented method for digital therapy for an individual in need thereof, the method comprising:
presenting the individual with a digital maze on a personal electronic device;
wherein the personal electronic device provides sensory modality input selected from: visual, auditory, tactile, or a combination thereof, to enable the individual to navigate the digital maze from a starting point to a finishing point.

In one embodiment the invention provides a computer-implemented method comprising:
navigation of an individual in a digital maze on a personal electronic device;
wherein the individual uses sensory modality inputs selected from: visual, auditory, tactile, or a combination thereof, to complete said digital maze from a starting point to a finishing point.

In one embodiment the digital maze comprises at least one obstacle selected from: outer wall, inner wall, dead end, object, turn, interconnected paths, or a combination thereof. “Obstacles” are defined as any individual item comprised in the digital maze. In its simplest form the maze can have no obstacles, and the individual needs to navigate from a starting point to a finishing point. In some embodiments the finishing point is viewable from the starting point. However, the digital maze can be of any shape, such as a winding corridor, where the user needs to navigate from start to finish. Obstacles are generally placed in the digital maze to add complexity and/or difficulty to the maze, however, as will become clear, the mere placement of objects in the maze isn't the only factor that can add to the complexity and difficulty of the maze.

In one embodiment the sensory modality inputs are configured to assist said individual in completing said digital maze in the shortest time, shortest path, with fewest obstacle impacts, or a combination thereof. For example, a user may see the maze in a digital format (e.g., on a phone application) and can swipe the phone to navigate through the maze. Additionally, the user may receive auditory cues to, for example, inform the user how close he is to an obstacle. In some embodiments each obstacle will have its unique sensory cue e.g., vibration, melody, sound, etc. In one embodiment the user is at least partially blindfolded when navigating the digital maze.

In one embodiment digital maze is presented to the individual to be repeated at least once. In one embodiment the digital maze is repeated between 1 and 10 times. In one embodiment the digital maze is repeated between 1 and 50 times. In one embodiment the digital maze is repeated between 1 and 100 times. As the user familiarizes himself/herself with the maze, they will become more proficient in completing the maze. The user may repeat the maze of the same difficulty. Otherwise, the user may return to easier mazes, as will be explained. One aspect of the digital mazes is that they become increasingly more challenging to the user. Since the digital mazes are used in digital therapy interventions, the element of progression through mazes of increasing difficulty is added, in various embodiments.

In one embodiment the method further comprises generating a performance score upon completion of each digital maze by taking into consideration the time taken, path taken, the number of obstacle impacts of the individual, or a combination thereof, when navigating said at least one digital maze. Each maze may have different elements that need to be considered in order to generate a performance score. The examples provided to generate a performance score are for the purposes of example alone. Each performance score can be personalized according to the user's needs and requirements. For example, the performance score for a simple maze may be the same as for a difficult maze, but they are weighted differently, because the difficult maze was more challenging to complete. This is taken into account when assessing the user's general performance in the digital mazes. It is also particularly relevant in tracking the user's progress through the digital maze exercises, and deciding how to proceed next. In one embodiment the method further comprises generating a threshold performance score for the digital maze, above which the individual is no longer presented with the digital maze to complete. The examples provided to generate a threshold performance score are for the purposes of example alone. Each threshold performance score can be personalized according to the user's needs and requirements. Thus, in various embodiments, the method comprises repeating the digital maze until a threshold performance score is achieved. In one embodiment, even if a threshold performance score is achieved, the user can decide to continue repeating that digital maze. In one embodiment the threshold performance score is changed to make the completion of a maze more challenging. For example, a simple maze can be made more challenging by increasing the threshold performance score. This would mean that the user would, for example, need to complete the maze quicker, or along a more efficient path, etc.

In one embodiment the method further comprises the personal electronic device providing a plurality of the digital mazes wherein an increase in sensory substitution is exhibited for each digital maze in the plurality of said digital mazes, following the completion of each digital maze and/or said threshold performance score being achieved; and wherein the sensory substitution comprises the personal electronic device providing the at least partial substitution of at least one of said sensory modality inputs with at least one other of said sensory modality inputs.

In one embodiment the method further comprises repeating a plurality of said digital mazes, wherein following the completion of each digital maze and/or said threshold performance score is achieved, an increase in sensory substitution is exhibited for each subsequent digital maze in the plurality of said digital mazes, wherein the sensory substitution comprises the at least partial substitution of at least one of the sensory modality inputs with at least one other of the sensory modality inputs.

As mentioned above, and in one embodiment, the user can decide to repeat a digital maze even if he has completed it previously and/or he has achieved the threshold performance score.

In one embodiment the method further comprises the complete substitution of one of the sensory modality inputs with at least one other of the sensory modality inputs selected from:
visual to auditory and/or tactile;
auditory to visual and/or tactile;
tactile to visual and/or auditory.

In one embodiment the personal electronic device is configured to execute the navigation by means of touch gesture, motion gesture, voice commands, text input, camera and media interaction, sensor-based interactions, or a combination thereof. In one embodiment the touch gesture comprises: tapping, swiping, scrolling, pinching, dragging, double-tapping, or a combination thereof. In one embodiment the motion gesture comprises: tilting, shaking, rotating, body motion, waving, or a combination thereof.

As the individual navigates the maze, the sensory cues can change to direct him through. In one embodiment the auditory inputs are selected from: a change in pitch, change in loudness, change in tone, change in melody, change in rhythm, change in music or a combination thereof. For example, if the individual gets closer to a wall the sound may get louder, or change pitch. This is true if the individual moves closer to the wall, but it can also occur for a moving obstacle which moves towards or away from the individual. The auditory cue can change for each object, e.g., a particular melodic phrase for each individual object. The individual can then be asked about the presence of various objects in the maze. For example, once the digital maze is completed, the user can be asked to identify the objects (and how many of them there were) in the maze by use of auditory cues alone. As the individual progresses through the digital mazes, incorporating sensory principles, the user will recognize that specific sounds corresponding to specific objects. The user may be asked to draw a sketch of the maze afterwards, based on the user's navigation through it, using auditory cues alone. The same principle applies to tactile cues. For example, a particular tactile cue can correspond to different events in the digital maze e.g., approaching an obstacle, hitting an obstacle, nearing the finishing point, etc. A particular vibration can correspond to these digital maze events e.g., double-vibration, short vibration, long vibration, a particular vibration rhythm, etc.

In one embodiment the method further comprises at least one additional device selected from: health monitoring system, medical device, haptic device, external speakers, headphones, virtual reality set, augmented reality glasses/devices, biofeedback sensors, wearable activity trackers, smartphone, personal computational device, smart speakers, voice assistants, motion tracking sensor, virtual assistant systems, internet hub, or a combination thereof; wherein the at least one additional device is configured to transfer data between said individual, said personal electronic device, or a combination thereof. Further examples include, Transcranial Magnetic Stimulation (TMS) devices, and Deep Brain Stimulation (DBS) devices. In one embodiment the personal electronic device, the at least one additional device, or a combination thereof, are configured to provide said sensory modality inputs. In one embodiment the tactile input is a vibration.

In one embodiment the individual completes a plurality of digital mazes of increasing difficulty following the completion and/or achievement of a threshold performance score, for each digital maze. In one embodiment the method further comprises the personal electronic device providing a plurality of digital mazes of increasing difficulty following the completion and/or achievement of said threshold performance score, for each digital maze.

As will be understood, an individual can complete and repeat a single digital maze any number of times. That same digital maze can then be repeated using the ‘sensory principles’ of sensory inhibition, sensory substitution, sensory integration, or a combination thereof. This also applies to any maze of any difficulty. Thus, once an individual has completed a simple maze, and gone through the sensory inhibition/substation/integration, of varying levels, the individual can then do the same for other digital mazes, but of increased difficulty. The individual can also return to any of the digital mazes that were previously attempted and/or completed. For example, if the individual had taken a break from carrying out the digital interventions, the individual can return to the easier digital mazes which were easier to complete.

In one embodiment the method further comprises the personal electronic device providing an increase in sensory substitution exhibited for each of the digital maze of increasing difficulty: wherein said sensory substitution comprises the personal electronic device providing the at least partial substitution of at least one of the sensory modality inputs with at least one other of the sensory modality inputs.

In one embodiment the method further comprises the complete substitution of one of the sensory modality inputs with at least one other of the sensory modality inputs selected from:
visual to auditory and/or tactile;
auditory to visual and/or tactile;
tactile to visual and/or auditory.

Modifying the difficulty of the digital maze can come in many forms. It will be understood that the difficulty level of a particular maze is dependent on the individual navigating the digital maze. For example, different obstacles or different sensory substitutions will be more challenging to some individuals than others. However, guiding principles can be used to generally design the digital maze to be increasingly more difficult. In one embodiment the increasing of the difficulty is achieved by: randomly generating a digital maze of a different structure, increasing the path length, increasing the number of turns, increasing the number of obstacles, diversifying the types of obstacles, decreasing the path width, incorporating a time challenge, increasing the performance threshold, changing the sensory modality inputs, adding interactive elements, incorporating distractions, incorporating tasks, incorporating moving obstacles, or a combination thereof “Turns” refers to changing the path direction within the digital maze e.g., with a wall, or an obstacle. “Diversifying the types of obstacles” refers to the different types of obstacles e.g., placing more of one type of obstacle compared with another.

Increasing the performance threshold means that the individual needs to complete the digital maze more effectively, in a shorter time, with fewer crashes into obstacles, etc. Changing the sensory modality inputs refers to using the sensory principles to add complexity to the digital maze. For example, using clouding of the digital maze, means that the user must rely on auditory/tactile cues rather than vision. Or an auditory cue can be changed so that the user must figure out how to next proceed in the maze, given that change. The digital maze can be made into a game where the user needs to interact with an element in the digital maze. For example, the user needs to reach a particular location within the digital maze, before the user proceeds to the finishing point. The user may be asked to interact with various elements in the maze. For example, the user can be instructed to ‘carry’ one item at one point in the maze, and transfer it to another location in the maze. In various embodiments, these interactive tasks may rely on auditory cues alone, whereas navigation in the maze is visual. For example, a task may require the user to pick up an object and place it on the other side of the maze, but the only cue that the user receives about the object is a louder sound when the user is close to the object, a different sound when the user picks up the object, and another sound to direct the user to place the object at a particular location. These principles can be applied to any configuration of the digital mazes and the examples provided herein are not intended to be limiting in scope. In various embodiments, the digital mazes provide a platform from which to execute the sensory principles, primarily for digital interventions. Digital mazes are designed to test a user's memory and aims to improve it. As such, a user can be asked to recall elements that were experienced during navigation in the digital maze e.g., a portrait on a wall, or the position of an obstacle, etc.

The digital interventions described herein can be designed as games, and treatment plans can include gamification elements such as badges, scores, leader-boards, ranking, game currencies, and similar elements all triggering user's reward system, resulting in dopamine production and release. This outcome is beneficial on multiple fronts. It improves adherence to the digital interventions themselves as well as to the treatment plan as a whole. It also carries several therapeutic benefits. An increase in dopamine can modulate the immune system. In addition, in some diseases such as Parkinson's disease, where patients suffer from reduction in dopamine in the brain, and many patients are in fact treated with dopaminergic drugs, the increase in dopamine in the brain triggered by the digital interventions, might have a therapeutic effect on its own or in combination with such drugs. In one embodiment the method further comprises gamification elements. In one embodiment the gamification elements are selected from: badges, scores, leader-boards, ranking, game currencies, quests or missions, characters or avatars, virtual goods, social media features, experience points (XP), or a combination thereof.

In one embodiment the digital maze comprises between 1 and 1,000,000,000 obstacles. In one embodiment the digital maze comprises between 1 and 1,000,000 obstacles. In one embodiment the digital maze comprises between 1 and 1,000 obstacles. In one embodiment the digital maze comprises between 1 and 100 obstacles. In one embodiment the digital maze comprises between 1 and 10 obstacles. In one embodiment the digital maze comprises between 1 and 5 obstacles.

In one embodiment the obstacles are stationary, moving, or a combination thereof. In one embodiment the obstacles and stationary. In one embodiment the method further comprises delivering instructions to the individual before, during, after, or a combination thereof, said digital maze.

The digital mazes can be executed by an individual on any number of platforms and with any number of devices e.g., a personal electronic device. In one embodiment the personal electronic device is selected from: smartphones, tablets, wearable device, smart TVs, computers, laptops, E-readers, gaming consoles, smartwatches, fitness trackers, portable media players, digital cameras, virtual reality (VR) headsets, augmented reality (AR) device, portable GPS devices, portable Bluetooth devices, portable digital assistant, smart glasses and audio device or any combinations thereof.

In one embodiment the computer-implemented method is for use in a digital therapy. In one embodiment the computer-implemented method carries out at least one digital therapy intervention. In one embodiment the computer-implemented method is for use in a digital therapy intervention plan. Therefore, use of the digital mazes of the invention are incorporated into any of the digital therapy interventions disclosed herein.

The principles of sensory inhibition, sensory substitution, sensory integration, can be used for any of the therapeutic interventions disclosed herein.

In one embodiment the digital therapy comprises at least one digital therapy intervention comprising: sensory inhibition, sensory substitution, sensory integration, or a combination thereof, to said individual. Thus, the sensory principles employed for executing the digital mazes, and the implementation of sensory inhibition, sensory substitution, sensory integration, or a combination thereof, are understood as disclosed elsewhere herein.

In one embodiment each of the therapeutic interventions has a duration ranging between 0.5 min to 30 mins. In one embodiment each of the therapeutic interventions has a duration ranging between 10s to 60 mins.

Performance metrics may include, without limitation, task completion time, accuracy, movement smoothness, response latency, error rate, adherence frequency, session duration, vocal amplitude or pitch range, and engagement consistency. The system may monitor such metrics to generate progress indicators and determine whether a predetermined condition has been met. Predetermined conditions may include achieving a threshold score, plateauing improvement across multiple sessions, or completion of a target number of iterations or durations. These conditions may trigger modification, continuation, or termination of the therapeutic plan.

In one embodiment the predetermined condition is selected from: a predetermined number of iterations, a predetermined duration has elapsed, a predetermined number of therapeutic interventions have been completed, predetermined progress measurement, predetermined regression measurement, target threshold score achieved, and no further improvement is measured, the absence of a termination condition, or a combination thereof.

In one embodiment the predetermined condition is selected from: a predetermined number of iterations, a predetermined duration has elapsed, a predetermined number of therapeutic interventions have been completed, predetermined progress measurement, predetermined regression measurement, target threshold score achieved, and no further improvement is measured, or a combination thereof.

In one embodiment the methods of the invention continue even if no predetermined condition is met. For example, indefinitely, or until the patient can no longer perform therapeutic interventions.

In one embodiment the targeted interaction outputs are selected from: visual, auditory, tactile, or a combination thereof. In one embodiment at least one of the therapeutic interventions further comprises: sensory inhibition, sensory substitution, sensory integration, or a combination thereof

As used herein, and in one embodiment, the term “targeted interaction outputs” refers to sensory stimuli generated by the digital therapy delivery module, including visual (e.g., on-screen animations, dynamic cues), auditory (e.g., tones, speech prompts, feedback sounds), tactile (e.g., vibration pulses, haptic cues), or multimodal combinations thereof. These outputs may be delivered via personal electronic devices such as smartphones, tablets, wearables, or VR/AR systems, and are selected and modulated based on user profile and therapeutic objectives.

Any of the individual interventions described herein can comprise sensory inhibition, sensory substitution, sensory integration, or a combination thereof. As such, it is understood that when referring to a particular therapeutic intervention the ‘sensory principles’ apply. The “senses” referred to herein any of the following: visual, auditory, tactile, olfactory, and gustatory. In various embodiments, the senses refer to visual, auditory, and tactile. Examples of the sensory modalities and their corresponding inputs include: visual, auditory, tactile, gustatory, olfactory, proprioceptive and vestibular; all of which can be understood to be included in the sensory principles outlined in the present invention. “Senses” are also referred to “human sensory modality”, “sensory modality” or “modality” and can be used and understood interchangeably. The ‘outputs’ and ‘cues’ that correspond to these sensory modalities are understood interchangeably. Furthermore, when referring to the various modalities, different expressions can be used to describe them. For example: an image can be described as a visual cue/stimulus/image, and auditory cues can be referred to as ‘sounds’. An expert will understand that the use of these words are interchangeable according to the context in which they are referred to. In one embodiment “sensory inhibition” refers to the at least partial suppression of at least one human sensory modalities. Suppression refers to the implementation of where one modality is decreased or at least partially removed, e.g., covering one eye suppresses the visual capability of the individual; or lowering the volume of a melody reduces/suppresses the auditory input to the individual. In one embodiment the at least one human sensory modality output is selected from: visual, auditory, and tactile. As used in this context herein “output” refers to a sensory modality that is presented or delivered to the individual. As understood herein “sensory substitution” refers to the at least partial replacement of at least one human sensory modality output with at least one other human sensory modality output. In one embodiment the at least partial replacement of at least one sensory modality input with at least one other sensory modality input is selected from:
visual to auditory and/or tactile;
auditory to visual and/or tactile; and
tactile to visual and/or auditory.

The integration of more than one sensory modality output is achieved by combining sensory modality outputs. As understood herein, and in one embodiment, “integration” regarding the sensory modalities refers to the integration of the input of the modalities e.g., using visual cues and auditory cues together. In one embodiment sensory integration refers to the at least partial combination of at least two sensory modality outputs. In one embodiment the personal electronic device, the at least one additional device, or a combination thereof, are configured to provide sensory modality outputs to carry out the sensory inhibition, sensory substitution, sensory integration, or a combination thereof. In various embodiments the integration of the sensory modality outputs includes the following modalities: visual, auditory, and tactile. For example, a visual and auditory cue can be experienced together e.g., a particular sound (auditory) corresponding to a particular image (visual), and these can further be integrated with a vibration (tactile) sensation coupled with the auditory and visual cues such that the same event in time is experienced by the individual but with different modalities.

In one embodiment the method further comprises a conditioning stimulus delivered before, during, after, or a combination thereof, the administering of the at least one medicinal agent or at least one of the therapeutic interventions. In some embodiments conditioning stimulus is selected from: visual, auditory, tactile, gustatory, olfactory, proprioceptive, and vestibular, or a combination thereof. As used herein “conditioning” (or “conditioning stimulus”) refers to the process of associating a particular cue or stimulus with the administration of a drug or medication in order to promote positive effects or outcomes. In one embodiment the conditioning stimulus is delivered before, during, after, or a combination thereof, the administering of the at least one medicinal agent, at least one of the therapeutic interventions, or a combination thereof. When therapeutic interventions are delivered in conjunction with the administration of medicinal agents, the combination may produce a priming effect, wherein the therapeutic activity sensitizes or prepares the individual's physiological or cognitive systems to respond more effectively to the pharmacological treatment, thereby enhancing overall therapeutic efficacy

In certain embodiments, the conditioning stimulus is used to reinforce associative learning between a sensory cue and the therapeutic or pharmacological effect. For example, a distinct light pulse, vibration, or tone may be paired with drug administration or with the initiation of a digital intervention, enabling conditioned therapeutic responses and supporting neurobehavioral reinforcement even in the absence of the active stimulus. This associative pairing may enhance expectancy, adherence, and long-term consolidation of treatment gains.

In one embodiment the Parkinson's disease-related markers comprise any of the following selected from: Unified Parkinson's Disease Rating Scale (UPDRS) score, motor symptoms, tremor amplitude, gait symmetry, or reaction-time variability, cognitive function, autonomic function, fatigue, sleep-related markers, sensory or olfactory function, biochemical markers, molecular markers, dopamine levels, α-synuclein, neuroinflammatory markers, oxidative stress markers, neurotrophic factors, pro-inflammatory cytokines, electrophysiological markers, and imaging-derived measures of dopaminergic function, basal ganglia structure, or functional connectivity, or a combination thereof.

Modulation of Parkinson's disease-related markers as described herein may be evidenced by measurable improvements across one or more standardized instruments, including but not limited to: the Movement Disorder Society-Unified Parkinson's Disease Rating Scale (MDS-UPDRS), Beck Depression Inventory (BDI-II), cognitive testing (e.g., Stroop, Trail-Making), gait analysis metrics, or imaging-derived neural connectivity measures. Biological improvements may also be detected through reductions in α-synuclein aggregation, normalization of dopaminergic function on PET or rs-fMRI, or changes in inflammatory and oxidative stress biomarkers.

### Systems of the Invention

In one embodiment the invention provides a system to implement the methods of the invention. In one embodiment the system is defined as one that carries out the methods of the invention.

In one embodiment the invention provides a digital therapeutic system for modulating one or more Parkinson's disease-related markers, symptoms, or disease progression in an individual in need thereof, the system comprising:
a data-input module configured to onboard the individual and dynamically monitor and store data related to the individual throughout a therapeutic plan; and
a digital therapy delivery module comprising a processor configured to generate the therapeutic plan comprising therapeutic interventions selected from: at least one motor skills intervention, at least one psychological intervention, at least one cognitive intervention, or a combination thereof;
wherein the digital therapy delivery module is further configured to convert the therapeutic plan into a plurality of targeted interaction outputs for presentation to the individual and track the individual's responses.

In one embodiment the invention provides a digital therapeutic system for modulating one or more Parkinson's disease-related markers, symptoms, or disease progression in an individual in need thereof, the system comprising:
a data-input module configured to onboard the individual and dynamically monitor and store data related to the individual throughout a therapeutic plan; and
a digital therapy delivery module comprising a processor configured to deliver the therapeutic plan comprising therapeutic interventions selected from: at least one motor skills intervention, at least one psychological intervention, at least one cognitive intervention, or a combination thereof;
wherein the digital therapy delivery module is further configured to convert the therapeutic plan into a plurality of targeted interaction outputs for presentation to the individual and track the individual's responses.

In one embodiment the digital therapy delivery module comprises software executable on a personal electronic device. In one embodiment the software is in the form of an application for a personal electronic device. In one embodiment the processor is further configured to generate the therapeutic plan.

In one embodiment the data-input module is configured to receive data from multiple data acquisition sources in real-time, wherein the data comprises one or more of: Electronic Medical Records (EMR) data, motion sensor data, position sensor data, environmental sensors data, audio sensors data, location sensors data, audio recording data, speech recognition data, gaze-tracking data, touch interaction data, physiological sensor data, optical sensor, imaging sensors, self-report data, or a combination thereof.

In one embodiment the digital therapy delivery module comprises any of the following selected from: a personal electronic device, a graphical user interface, audio interface, haptic interface, or a combination thereof. In one embodiment the data-input module is further configured process the data and adapt the therapeutic plan.

As used herein, the term “personal electronic device” is understood to be any electronic device used by a user to carry out any part of the digital intervention. Thus, although it is often a ‘personal’ device, it is also understood to include devices that are not directly owned by the user, but those that are used/synced for the user to implement the digital intervention. The following are examples of personal electronic device, but are not limited to: smartphones, tablets, wearable device, smart TVs, computers, laptops, E-readers, gaming consoles, smartwatches, fitness trackers, portable media players, digital cameras, virtual reality (VR) headsets, augmented reality (AR) device, portable GPS devices, portable Bluetooth devices, portable digital assistant, smart glasses and audio device or any combinations thereof. In various embodiments the personal electronic device is a mobile computing device.

In one embodiment the processor is further configured to dynamically monitor any of the following factors of the individual selected from: Electronic Medical Records (EMR) data, motor attributes, psychological attributes, cognitive attributes, pharmacological attributes, and drug intake parameters, or a combination thereof, throughout the implementation of the therapeutic plan, and generate a dynamic user profile.

In one embodiment the processor is further configured to recursively adapt the therapeutic plan based on the dynamic user profile, performance metrics, and the individual's responses.

The digital therapy delivery module may, in certain embodiments, not only execute but also generate or update the therapeutic plan based on data received from the data-input module. Both modules may communicate bidirectionally via secure, encrypted channels to ensure real-time synchronization and data integrity. The data-input module may preprocess sensor data, identify anomalies, and update the dynamic user profile accordingly. The system may store anonymized session data locally or in cloud infrastructure for longitudinal analysis, algorithm refinement, or regulatory compliance. In one embodiment the data-input module is configured to receive and synchronize multimodal data streams from multiple data acquisition sources in real-time or near-real-time, wherein the multimodal data streams comprise two or more of: motion sensor data, speech recording data, gaze-tracking data, touch interaction data, physiological sensor data, or self-report data. In one embodiment the processor is further configured to apply time-alignment algorithms to synchronize data streams with different sampling rates into a unified temporal framework. In one embodiment the processor is further configured to generate a unified therapeutic response model by integrating performance data across motor, cognitive, psychological, and pharmacological domains, and wherein the processor uses the unified therapeutic response model to optimize the therapeutic plan. In one embodiment the unified therapeutic response model comprises a computational model that represents interdependencies between different therapeutic domains using one or more techniques selected from: correlation matrices, regression models, neural network architectures, Bayesian networks, state-space models, or dynamic systems models.

In one embodiment the processor is further configured to dynamically monitor any of the following factors of the individual selected from: Electronic Medical Records (EMR) data, motor attributes, psychological attributes, cognitive attributes, pharmacological attributes, and drug intake parameters, or a combination thereof, throughout the implementation of the therapeutic plan.

In one embodiment the processor is further configured to generate a dynamic user profile, and recursively adapt the therapeutic plan based on the dynamic user profile, performance metrics, and the individual's responses.

In one embodiment the processor is further configured to recursively adapt the therapeutic plan based on the dynamic user profile, performance metrics, and the individual's responses.

In one embodiment the recursive adapting of the therapeutic plan comprises using the unified therapeutic response model to: identify which intervention types yield the strongest therapeutic response for the individual; determine optimal timing for each intervention type based on time of day and pharmacological state; balance intervention types to address multiple symptom domains; and adjust intervention parameters based on predicted cross-domain effects. For example, if mindfulness-based emotional-regulation exercises are predicted to reduce freezing-of-gait episodes, the system may increase their scheduling before high-mobility routines to exploit the cross-domain therapeutic effect.

In some embodiments, the digital therapeutic system is further configured to comprise gamification elements. For example, gamification elements are configured to enhance user engagement, motivation, and adherence to the therapeutic plan. These elements may include the awarding of digital badges, points, levels, or other forms of recognition based on the individual's progress, consistency, or achievement of therapeutic milestones. The system may also provide rewards or incentives, such as unlocking new content, personalized feedback, or virtual achievements. Additionally, the system may include features for sharing progress with caregivers, clinicians, or peer support networks, and may support synchronization across multiple devices or platforms to ensure continuity of experience and data integrity.

As understood herein “software application” is a program that runs on a device. In various embodiment the “software application” is a computer program which performs specific tasks or functions on a device. In embodiment the software application is an app on a smartphone.

### Methods for Modulating One or More Huntington's Disease-Related Markers

Huntington's disease (HD) is a fatal, hereditary neurodegenerative disorder caused by a CAG trinucleotide repeat expansion in the HTT gene on chromosome 4. This mutation results in the production of an abnormal, toxic huntingtin protein that accumulates within neurons, particularly in the striatum (caudate and putamen), leading to progressive neurodegeneration. Clinically, HD presents as a triad of progressive psychiatric, cognitive, and motor symptoms.

Neuroinflammation contributes to HD pathogenesis through early and sustained activation of microglia and astrocytes, which release pro-inflammatory cytokines such as TNF-α, thereby exacerbating neuronal dysfunction and degeneration. Anti-inflammatory and immunomodulatory strategies are employed herein for disease-modifying therapies in HD.

Huntington's disease and Parkinson's disease share several mechanistic and clinical characteristics: both involve basal ganglia dysfunction and dopamine dysregulation, though in opposite directions. Parkinson's disease is marked by slowness, rigidity, and tremor (hypokinetic symptoms), while Huntington's disease manifests as involuntary movements and chorea (hyperkinetic symptoms), which in advanced stages may progress toward rigidity and bradykinesia resembling Parkinsonian features. Dopamine signaling is disrupted in both disorders, with Parkinson's disease reflecting dopamine deficiency due to degeneration of substantia nigra neurons, and Huntington's disease demonstrating early dopamine excess followed by later-stage depletion.

Current pharmacological approaches include vesicular monoamine transporter 2 inhibitors such as tetrabenazine or deutetrabenazine, which alleviate chorea; dopaminergic and glutamatergic modulators that rebalance neurotransmission; sigma-1 receptor agonists such as pridopidine that support neuronal homeostasis; and various psychotropic agents including selective serotonin reuptake inhibitors, antipsychotics, and GABAergic modulators to address psychiatric manifestations. More recently, disease-modifying strategies have emerged, including antisense oligonucleotides directed to huntingtin mRNA, small-molecule splicing modulators of HTT expression such as PTC518 or votoplam, RNA interference agents that silence huntingtin transcripts, and adeno-associated viral (AAV) or other viral-vector-mediated gene therapies such as AMT-130. Additional modalities include CRISPR-based or allele-selective gene-editing therapies, as well as neuroprotective, mitochondrial, oxidative-stress, and microglial-modulating agents designed to preserve neuronal function and limit inflammatory injury.

The present methods complement these therapeutic strategies by attenuating neuroinflammation through inhibition of pro-inflammatory cytokine release, including TNF-α, and by reducing the generation of reactive oxygen species, thereby improving neuronal resilience. Similar benefits on mental health outcomes occur, consistent with effects observed in patients with subjective cognitive decline and Parkinson's disease treated using the present methods.

Based on data from Example 2, supporting the effects of non-pharmaceutical interventions in PD, comparable and corresponding effects are applicable to HD, which integrates motor, mental, and cognitive components. This multimodal approach is thus equivalent but for the reduction in the UHDRS (Unified Huntington's Disease Rating Scale) score.

Non-pharmacological interventions in HD address motor and balance difficulties, cognitive decline, psychiatric and behavioral symptoms and speech, swallowing, and nutritional challenges, as well as caregiver and psychosocial needs. These multidisciplinary interventions, delivered by physiotherapists, occupational and speech therapists, psychologists, dietitians, and social workers, are most effective when introduced early and combined with pharmacologic and supportive therapies, maximizing functional preservation and quality of life

In one embodiment the invention provides a method for modulating one or more Huntington's disease-related markers, symptoms, or disease progression in an individual in need thereof, the method comprising:
determining a therapeutic plan to be delivered to the individual, wherein the therapeutic plan comprises therapeutic interventions selected from: at least one motor skills intervention, at least one psychological intervention, at least one cognitive intervention, or a combination thereof;
converting the therapeutic plan into a plurality of targeted interaction outputs for presentation to the individual; and
adaptively delivering the therapeutic plan, tracking the individual's responses and associated performance metrics, and continuing until a predetermined condition is met.

In various embodiments, the methods, systems, and therapeutic workflows described herein for Parkinson's disease are applicable to Huntington's disease. The same features and operational steps may be used for modulation, disease modification, treatment, monitoring, personalization, or progression management in Huntington's disease. The Parkinson's-related embodiments are incorporated herein by reference for use in Huntington's disease. In further embodiments, adjustments may be made to account for differences in symptom patterns, clinical presentation, therapeutic goals, or other disease-specific factors relevant to Huntington's disease.

Likewise, and in various embodiments, the methods, systems, and therapeutic workflows described herein for Parkinson's disease are applicable to any of: Alzheimer's disease, amyotrophic lateral sclerosis (ALS), multiple system atrophy (MSA), progressive supranuclear palsy (PSP), corticobasal degeneration (CBD), frontotemporal dementia (FTD), essential tremor, dystonia, Tourette syndrome, spinocerebellar ataxia, multiple sclerosis (MS), lupus-related neuropsychiatric syndromes, Wilson's disease, neuroacanthocytosis, and Rett syndrome.

In one embodiment, the term “a” or “one” or “an” refers to at least one. In one embodiment the phrase “two or more” may be of any denomination, which will suit a particular purpose. In one embodiment, “about” or “approximately” may comprise a deviance from the indicated term of +1%, or in some embodiments, −1%, or in some embodiments, ±2.5%, or in some embodiments, ±5%, or in some embodiments, ±7.5%, or in some embodiments, ±10%, or in some embodiments, ±15%, or in some embodiments, ±20%, or in some embodiments, ±25%.

Those skilled in the art to which this invention pertains will readily appreciate that numerous changes, variations, and modifications can be made without departing from the scope of the presently disclosed subject matter, mutatis mutandis.

### EXAMPLES

### Example 1

### Examples of Non-Pharmacological Therapies for PD

Given the chronic and progressive nature of Parkinson's disease (PD), comprehensive management often involves a combination of pharmacological and non-pharmacological therapies. Non-pharmacological therapies for PD aim to improve both motor and non-motor symptoms and enhance overall quality of life.

Regular exercise including aerobic, strength, and balance training, is one of the most effective non-pharmacological interventions for PD. Clinical studies support the benefits of improving physical function, strength, gait speed, and balance, as well as non-motor functions like sleep and cognition.

Physiotherapy (including hydrotherapy and other movement-based therapeutic approaches) can also be valuable in addressing movement difficulties, enhancing posture, and managing freezing of gait.

Occupational therapy helps PD patients maintain independence in daily activities.

Speech therapy addresses speech difficulties, which can include speaking softly, using a monotone voice, slurring words, mumbling, and stuttering. Speech therapy techniques can improve speech volume and clarity, although adherence to vocal exercises is crucial for maintaining the benefits over time.

Finally, psychosocial support, including counseling, cognitive-behavioral therapy (CBT), and support groups, can help patients and caregivers cope with the emotional challenges of PD, and specifically with depression. Stress management techniques, such as mindfulness meditation, have also shown to reduce anxiety and improve mood, contributing to better overall quality of life.

### Example 2

### Study Design and Participants

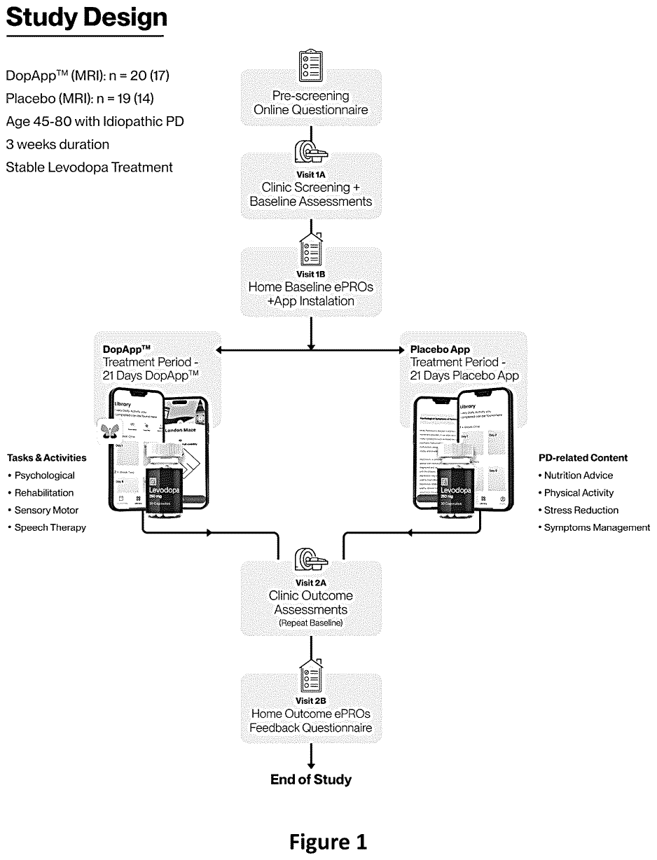

In the present study participants were recruited through social media and patient advocacy groups. Eligible participants, aged 45-80 years old with a recorded PD diagnosis and treated with stable daily regimens of levodopa (stable regimen of 150-1500 mg/day for ≥30 days with a maximum of five doses per day), were randomly allocated to either the DopApp™ or placebo app for three weeks (FIG. 1). Randomization was performed in a 1:1 ratio with a block size of 4, using a computer-generated randomization table. PwP with additional major unstable neurologic, psychiatric or medical conditions were excluded. Participants taking other PD drugs were included in the study, and LD equivalence doses were calculated. All study personnel were blinded to treatment allocation, except for the technical staff responsible for app installation and onboarding, and the technical helpdesk when applicable.

### Treatment

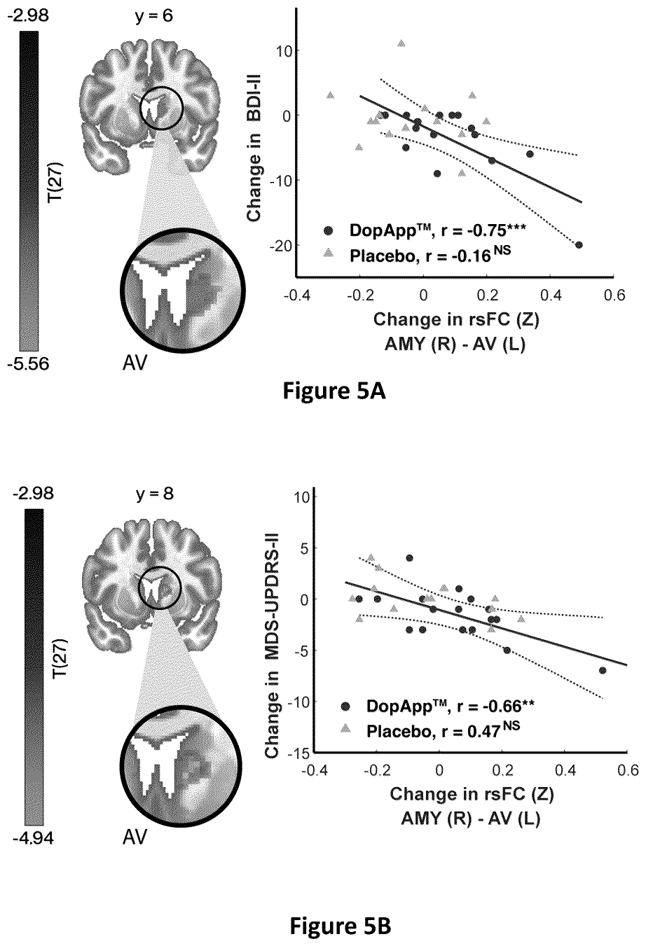

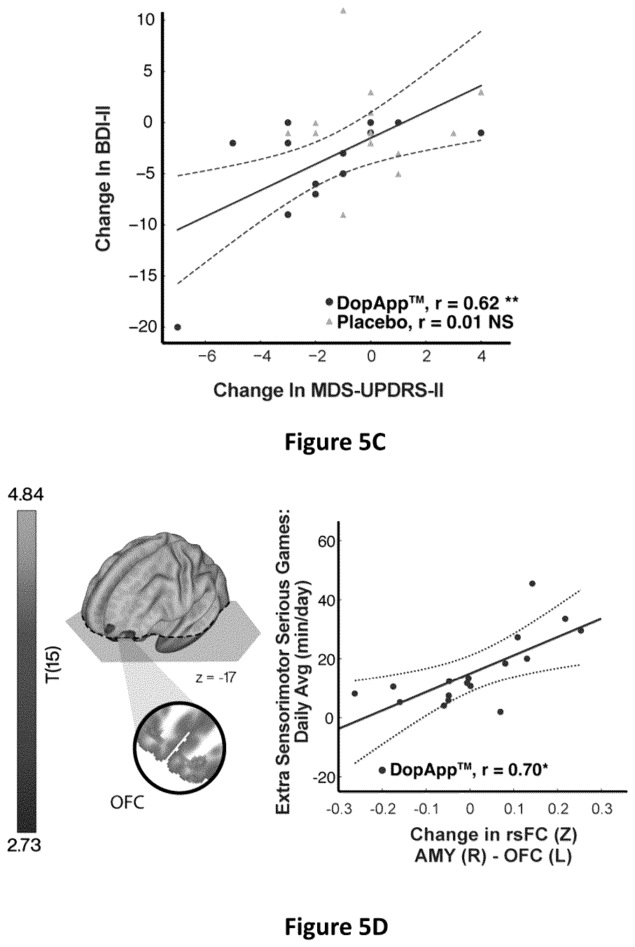

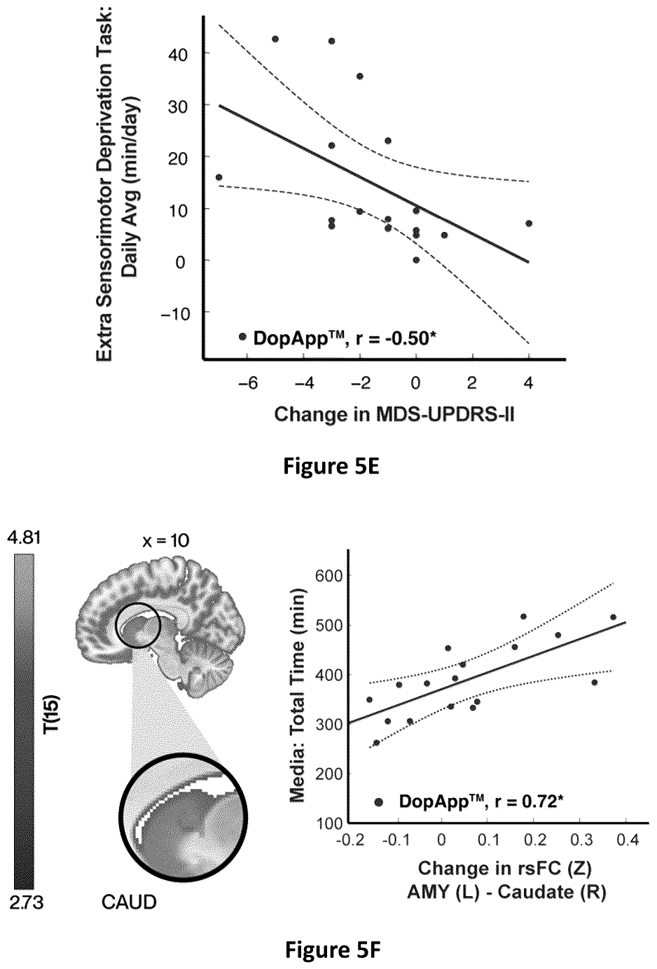

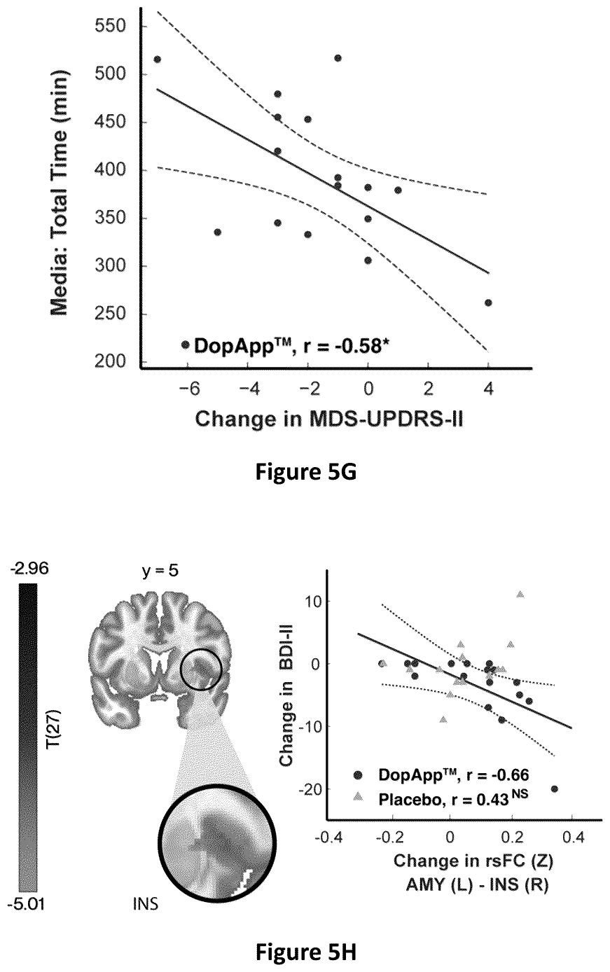

The DopApp™ intervention is a comprehensive multimodal program integrating cognitive, psychological, and rehabilitation-focused exercises across well-established disciplines. It incorporates principles of multisensory (vision, audio) integration, sensory substitution, and masking. These techniques were digitized and delivered through short interactive videos, audio, and engaging games. DopApp™ interventions were structured into 21 daily routines and included the following components: (i) Digital adaptations of relevant fine-motor skills, physiotherapy, occupational therapy, speech therapy, and dance exercises. (ii) A virtual audio-visual spatial memory navigation exercise based on the principles of sensorimotor integration and gradual visual masking. (iii) Psychological interventions adapted from cognitive behavioral therapy (CBT), acceptance and commitment therapy (ACT), mindfulness-based stress reduction (MBSR), mindfulness-based cognitive therapy (MBCT), guided imagery, psychoeducation, and attention training. (iv) Daily walking, including at least 15 minutes of scheduled walking (see FIGS. 5A-5D).

The app utilized adaptive algorithms to dynamically adjust task difficulty based on real-time performance metrics, engagement, and progression. The placebo app had a similar appearance to the DopApp™, and onboarding steps were identical. The placebo intervention protocol consisted of a daily regimen featuring PD-related content such as nutritional guidance and the general benefits of physical activity (FIG. 1). All study participants received daily new content, along with app usage reminders, as well as access to an on-demand library of previously completed interventions.

### App Analysis

To evaluate user engagement with the DopApp™ intervention, all app interactions were automatically logged, including timestamps and activity-type identifiers. These data were analyzed to quantify usage patterns across the app's various components and to distinguish between content completed as part of the structured protocol and content accessed voluntarily.

For analysis purposes, app activity was categorized into two main functional domains: (i) Emotion regulation (via media content)—This category included prerecorded video and audio materials such as guided mindfulness sessions, psychoeducational content (the majority of the content), and therapeutic exercise videos in areas such as physical therapy, speech therapy neuro-dance and occupational therapy. (ii) Sensorimotor (via serious sensorimotor games)—activities that integrate sensory input with motor response. All sensorimotor games were interactive tasks requiring active user participation. Users had to engage with the task and meet a minimum performance threshold to progress through the daily protocol. Each game provided immediate feedback, such as scores, star ratings, or progression indicators. Games targeted motor, sensorimotor, or speech-related functions, and included tasks designed to train fine motor skills, vocal control, coordination, and spatial processing. Within this category, the sensorimotor-deprivation intervention, a virtual spatial memory navigation exercise, was the most prominent and was therefore also analyzed separately.

Content was also classified by use type: (i) Protocol-driven use (required): engagement with daily assigned sessions within the three-week intervention period. (ii) Self-initiated use (extra): any additional, self-initiated engagement with content through the on-demand content library. Specifically, self-initiated games measure, which reflect involvement in cognitively demanding and reward-based tasks, was used as a motivation score.

Each participant's usage data was analyzed using three summary engagement metrics: (i) Exposure-Total Time (minutes): the cumulative time spent using a given content type. This reflects overall exposure and adherence to that component. (ii) Daily-dose: Average Daily Usage (minutes/day): the mean time spent per day on active days. This captures daily engagement or daily-dose for each content type. (iii) Usage consistency-Number of Days: the total number of calendar days in which a participant engaged with the content. This metric was only calculated for self-initiated use, as protocol-driven use occurred consistently across all 21 intervention days by design. Therefore, usage consistency reflects voluntary long-term engagement and adherence beyond the structured protocol

Participants were assessed at baseline and at the end of study by a blinded rater, from an external medical services center, with the same clinician conducting both assessments for each participant. The primary therapeutic outcome was the change from baseline in the MDS-UPDRS total score (sum of parts I, II & III) compared with placebo. MDS-UPDRS part III assessments were conducted in the ON state as determined by the patient's subjective experience of motor function improvement after taking the medication. Additional outcomes included changes from baseline in: MDS-UPDRS sub-parts (part II, part III, sum of parts II & III, and item clusters related to speech and fine motor function), the Parkinson's disease sleep scale (PDSS2), PD Questionnaire 39-item (PDQ-39), Voice Handicap Index (VHI), Perceived Stress Scale (PSS-10), Brief State and Trait Anxiety Inventory (STAIT-5/STAIS-5), Beck's Depression Inventory II (BDI-II), Mental Health Continuum Short Form (MHC-SF), Brief Resilient Coping Scale (BRCS), and the Trail Making Test (TMT). Daily app engagement was recorded, and participants completed an exit feedback questionnaire.

### Brain Imaging

Brain imaging MRI scans were performed on a MAGNETOM Prisma 3T Scanner, configured with 64-channel receiver head coils (Siemens Healthcare, Erlangen, Germany), at the Ruth and Meir Rosental Brain Imaging Center (MRI), Reichman University. Due to dopaminergic effects on rsFC, PD medications were administered two hours prior to MRI scans. The MRI protocol included the following sequences: Two runs of rs-fMRI scans (300 volumes, 9:28 min) were acquired using a multi-band echo planar imaging sequence, CMRR EPI 2D, Scan parameters: TR: 1,870 ms, TE: 30 ms, flip angle: 75°, voxel size: 3.0×3.0×2.0 mm, FOV: 192, number of slices: 58, axial slices parallel to the AP-PC plane. During scanning, participants were asked to remain still and relaxed, with their eyes fixated on a cross, and without deliberately thinking of anything. Foam pads and earplugs were employed to reduce head motion and scanning noise. Structural T1-weighted MRI scans were acquired for co-registration purposes using a T1-weighted 3D magnetization-prepared rapid gradient-echo (MPRAGE) sequence in a sagittal plane with 1 mm isotropic resolution. Sequence parameters: TR: 2,000 ms, TE: 1.9 ms, flip angle: 9°, TI: 920 ms, FOV: 256×256, and 176 contiguous slices. The MRI protocol also included T2-fluid-attenuated inversion recovery (FLAIR) sequences, using standard parameters for clinical brain evaluation.

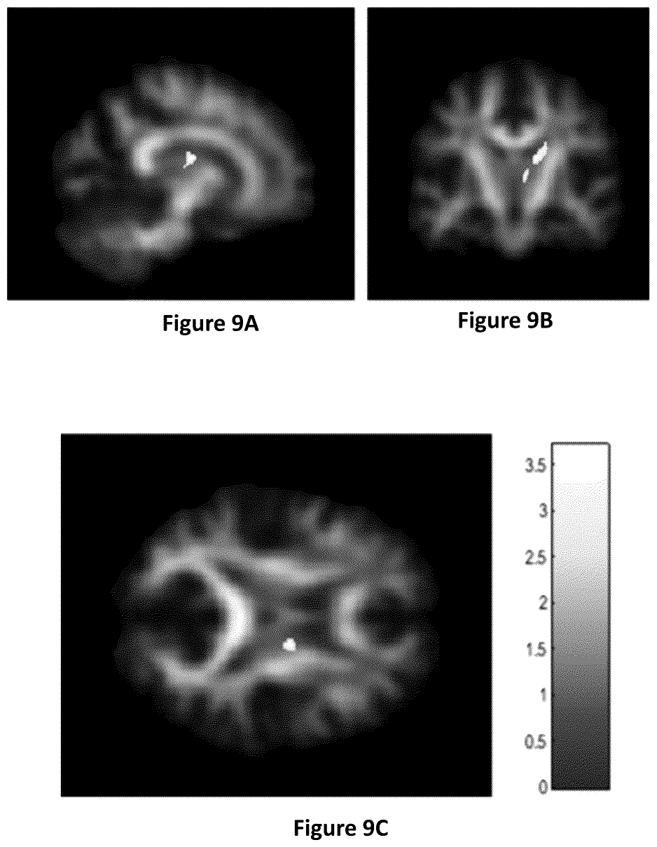

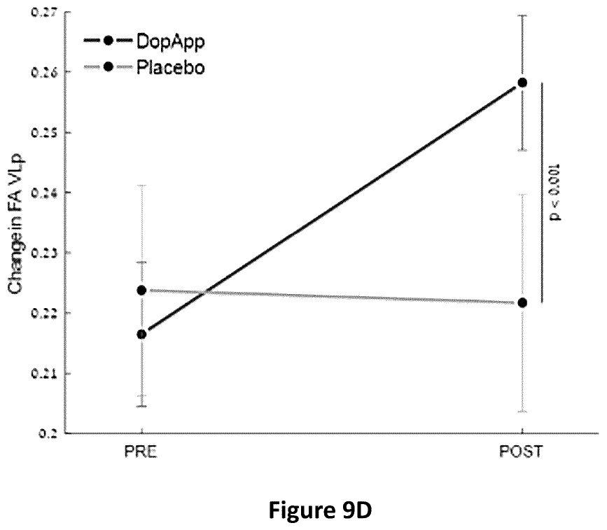

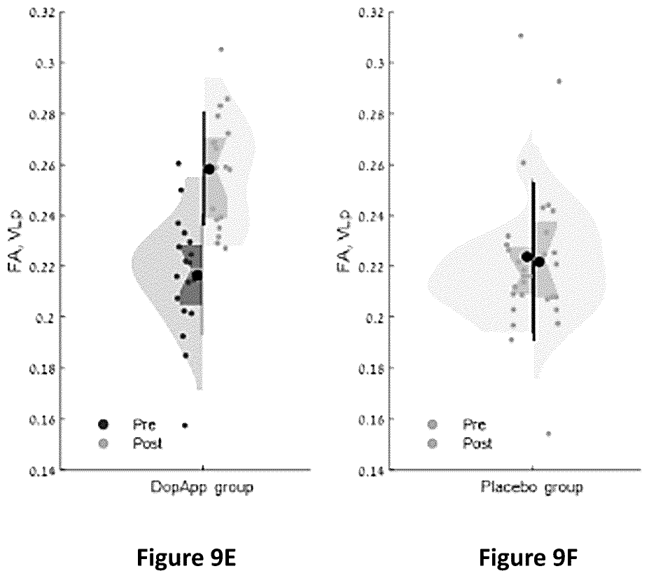

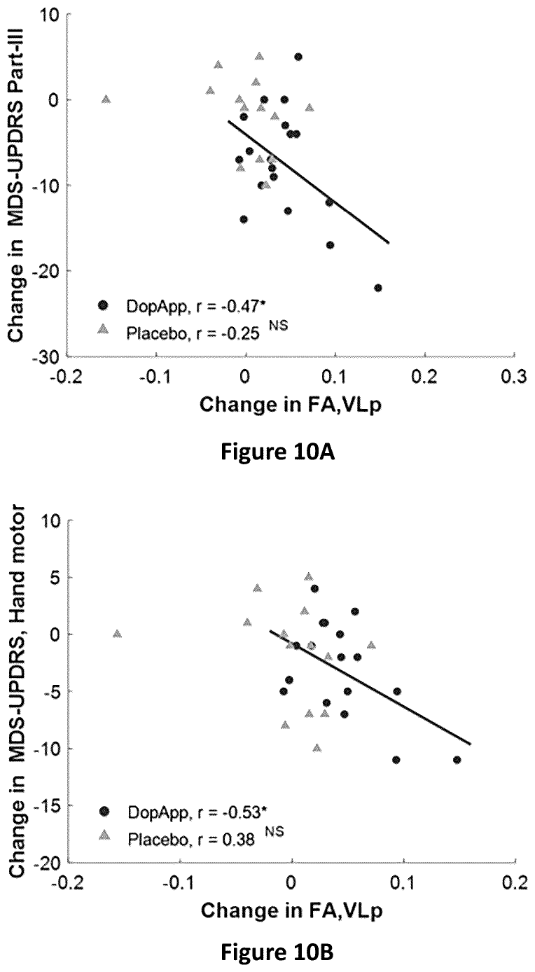

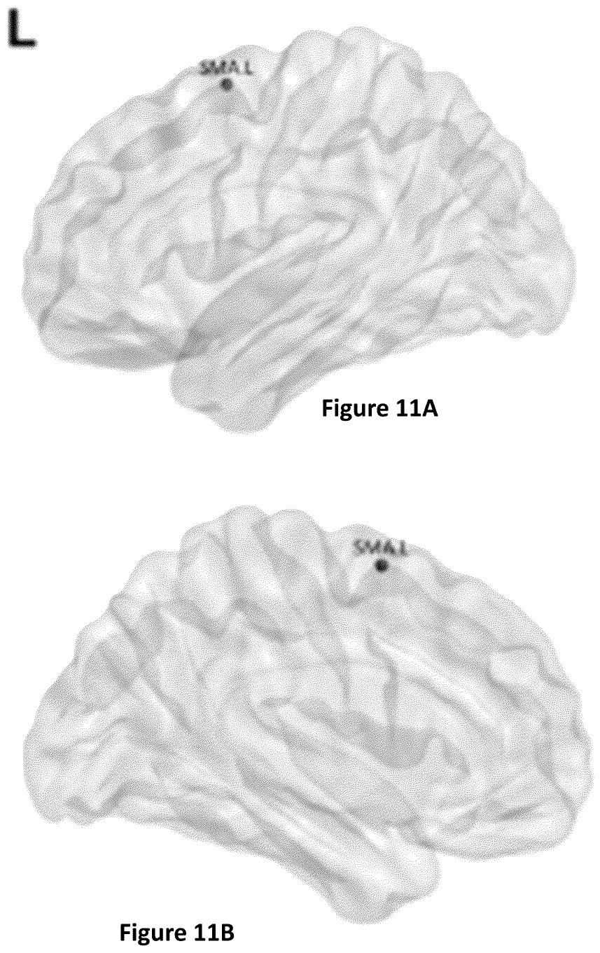

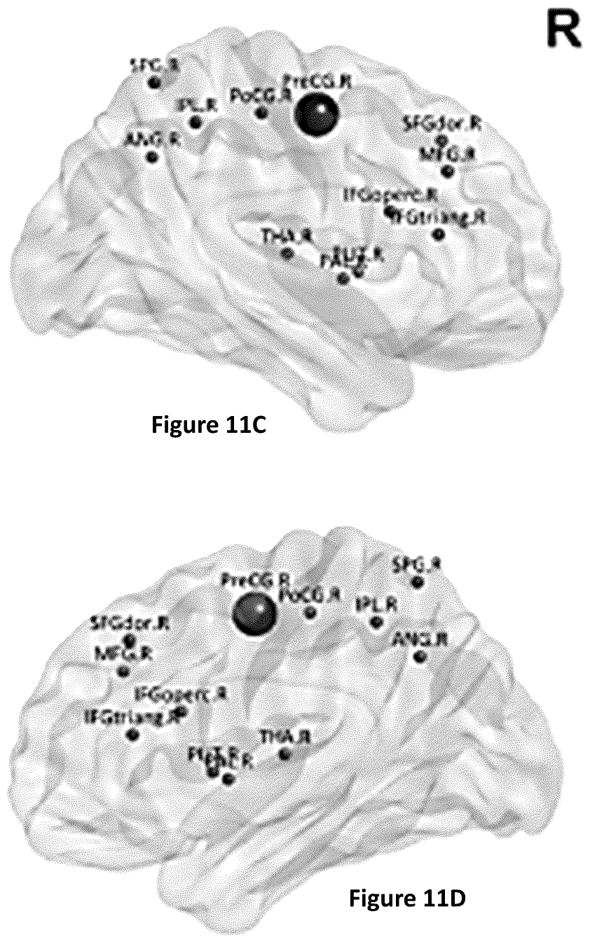

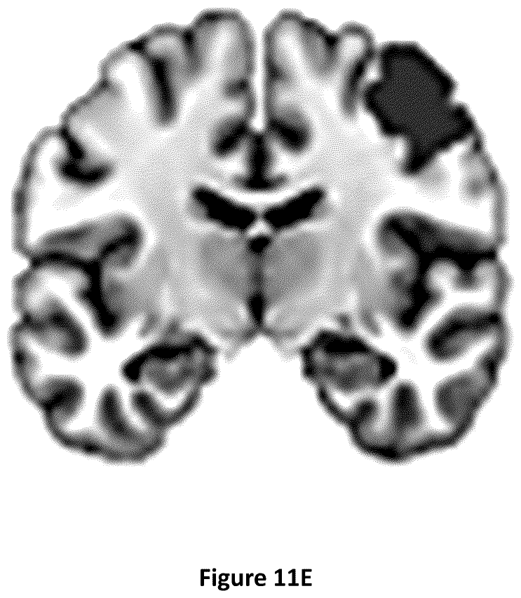

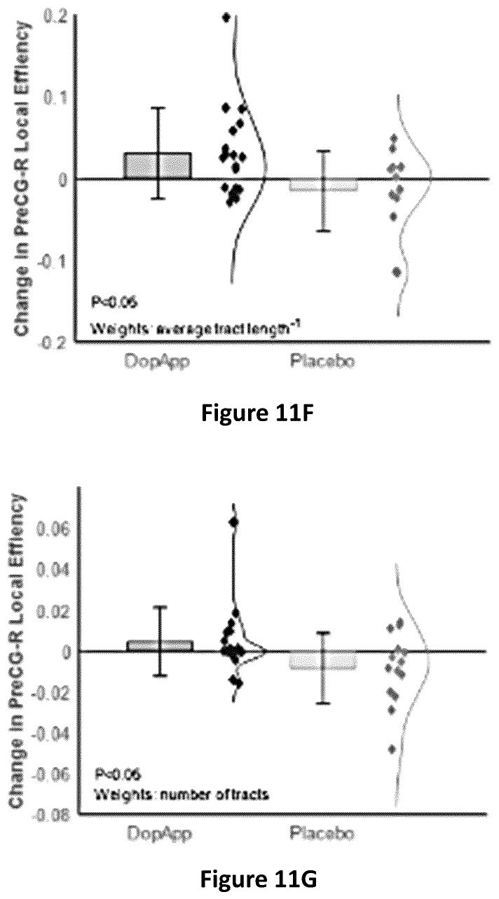

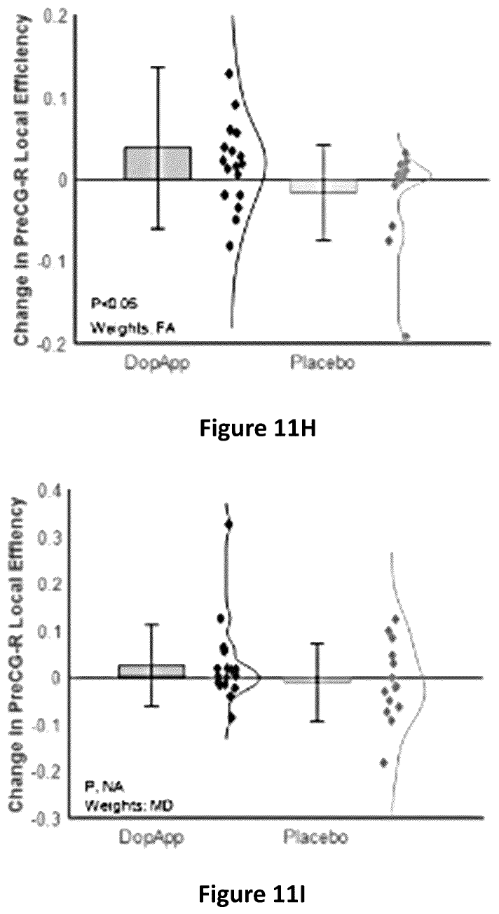

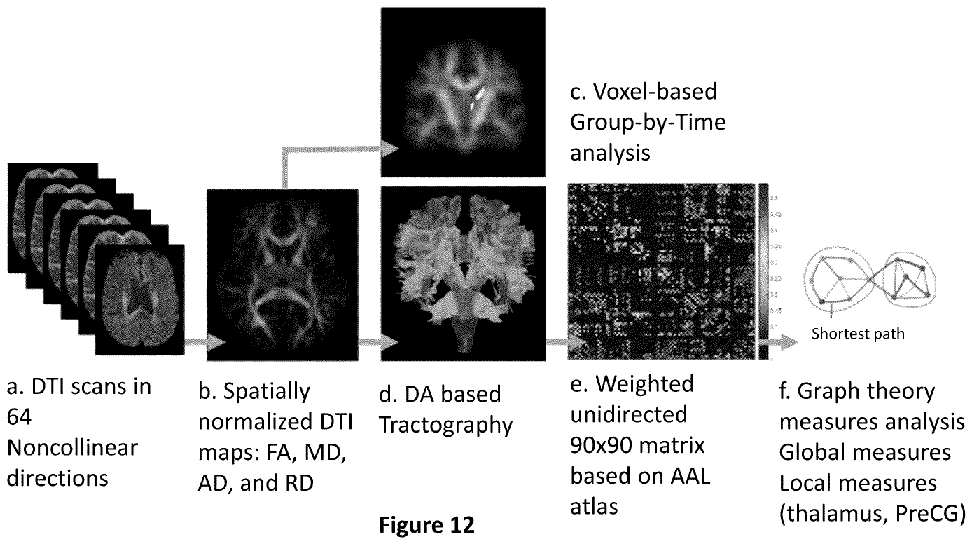

In one example, a multimodal digital intervention combined with levodopa in people with Parkinson's disease induced enhanced axonal integrity and structural efficiency of thalamocortical pathways reflecting microstructural thalamo-motor connectivity restoration process. FIG. 12 shows a DTI image processing pipeline and FIGS. 9A-9F show voxel-based Diffusion Tensor Imaging (DTI) for DopApp and placebo intervention groups.

### BOLD Data Preprocessing and Seed Selection

RsFC analysis was carried out using the CONN (RRID: SCR_009550), as implemented using statistical parametric mapping software SPM12. Functional volumes preprocessing pipeline included realignment with correction of susceptibility distortion interactions, slice timing correction, outlier detection, direct segmentation, and MNI-space normalization, with a resolution voxel size of 2.0×2.0×2.0 mm, and spatial smoothing (8 mm FWHM Gaussian kernel) steps. A component-based noise correction procedure (CompCor) approach was used to identify additional confounding temporal factors controlling for physiological noise, BOLD signal present in white matter, and head motion effects. Residual BOLD time series were then bandpass filtered at a frequency range of 0.008-0.09 Hz. Individual connectivity maps were generated using the seed-to-voxel approach. Bivariate correlation analysis was used to determine the linear association of the BOLD time series between the seed and significant voxel clusters. Fisher's Z transformation was applied to the correlation coefficients to satisfy normality assumptions. Finally, participants with head motions of>3 mm in any direction between volumes, rotations of>3° in any axis during scanning or mean framewise displacement (FD) scan-to-scan head-motion>0.5, in either the pre- or post-treatment maps, in both runs, were excluded from the dataset.

To elucidate the parallel neural mechanisms underlying the combined effects of standard levodopa treatment and the DopApp™ digital intervention on both motor and NMS, segregated thalamocortical circuits were investigated. These circuits were chosen due to the thalamus's role as a critical relay integrating BG output to the cortex. To assess the impact of sensorimotor training, rsFC was evaluated using a priori seed regions located in key ventral lateral thalamic nuclei, as defined by the extended Human Connectome Project-MultiModal Parcellation Atlas (HCPex). Specifically, ventral lateral anterior (VLa) was selected, ventral lateral posterior (VLp), and ventral posterolateral (VLP) nuclei of the thalamus, along with the primary motor cortex (M1), as regions of interest. The VL nuclei integrate motor control by receiving inputs from the BG, striatum, cortex and cerebellum, and projecting primarily to M1. These nuclei have been implicated in regulating PD motor symptoms.

To examine the limbic thalamocortical pathway, a priori seed regions were selected within thalamic subregions implicated in limbic processing, based on the HCPex atlas. These included bilateral seeds in the anteroventral thalamic nucleus (AV), the mediodorsolateral parvocellular nucleus (MDI), and the mediodorsomedial magnocellular nucleus (MDm). The AV nucleus, a principal component of the limbic thalamus, links memory and emotion circuits by interfacing with limbic regions of the default mode network (DMN), thereby supporting integrative information processing. The MD complex, through its extensive connections with the prefrontal cortex and other cortical areas, contribute to higher cognitive functions, including recognition memory and familiarity, via inputs from the perirhinal cortex, and play a role in modulating cortical network dynamics. In addition to these BG seed regions bilateral amygdala (Amyg) seeds were included to assess potential changes in functional connectivity associated with affective and emotional processing.

### Statistical Analysis

Efficacy analyses were performed for all participants who had met the a priori analysis inclusion criteria of completing ≥17 of the 21-day treatment. Two-tailed Welch's unequal variances 1-tests were performed to compare continuous variables between groups when a normality assumption was held according to Kolmogorov-Smirnov tests (P<0.05 was considered significant). Effect sizes were evaluated using Cohen's d method. Categorical data were compared using χ²/Fisher's exact tests. The response rate analysis was calculated as the proportions in each group and were compared using the Fisher's exact test.

Sensitivity analyses were also performed for the primary therapeutic outcome, including (i) exclusion of outliers (defined as participants having >3 median absolute deviations from the group median) (ii) inclusion of all study subjects who signed the informed consent, were randomized, completed the baseline evaluation and commenced treatment (n=42), (iii) non-parametric statistical tests using the Wilcoxon rank test, and (iv) a linear model with baseline MDS-UPDRS total score, number of years from PD diagnosis and the daily levodopa equivalent dose included as covariates.

To assess the relationship between variables, Pearson correlation analyses were performed within the DopApp™ group. For each variables pair, scatter plots were generated and overlaid with a linear regression line and its 95% confidence interval. All analyses were performed using R Statistical Software (v4.4.1; R Core Team 2024) and Python (v3.13.1; Python Software Foundation, 2024).

### Determination of Sample Size

Given the exploratory nature of this PoC, a target ollment of 40 PwP was planned. Although not powered to detect the MCID of 6.7 points, this sample was sufficient to identify a statistically significant between-group difference greater than 5.5 points in the primary endpoint (total MDS-UPDRS score) at 5% significance and 80% power.

### Imaging Statistical Analysis

At the group level, rsFC individual maps were analysed using the mixed design repeated measure ANOVA model to test the main interaction effect between time and group. RsFC was considered significant at joint-probability thresholds of 0.005 at the voxel level, and P<0.05 false discovery rate (FDR) corrected for multiple comparisons across the whole brain at the cluster level, with a minimum cluster size of 50 voxels. A bivariate group-level regression analysis with non-imaging covariates (e.g. psychological data, MDS-UPDRS) covariate model was used to identify global brain correlations. The Pearson correlation was then used to test for associations with the non-imaging covariates, where the REX toolbox was used to extract cluster connectivity values.

### Results

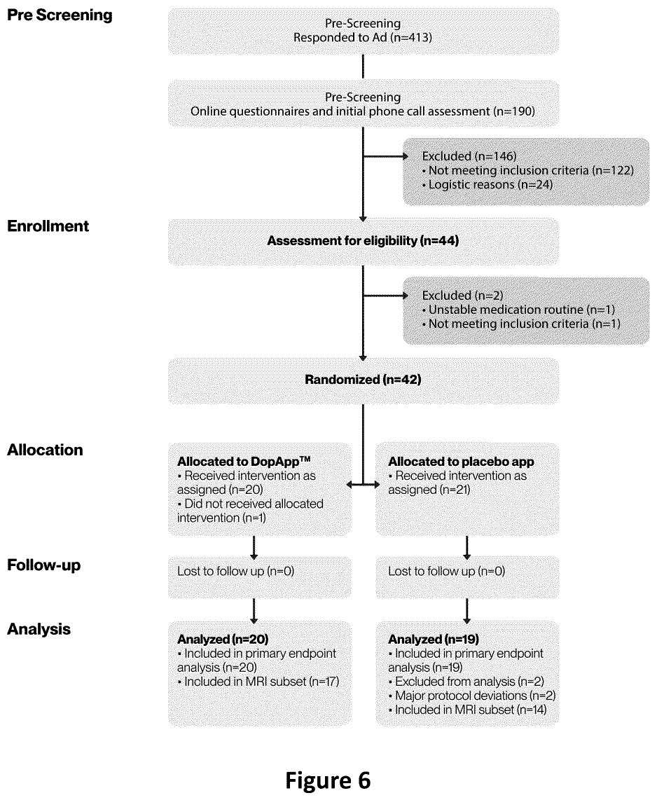

Patients. A total of 190 individuals responded to the study advertisement and answered the pre-screening questionnaire. Forty-two met the eligibility criteria and were subsequently enrolled and randomized to the study procedures. Of these, one participant from the DopApp™ group withdrew consent, and two participants from the placebo group were excluded due to major protocol deviations (see FIG. 6). Thirty-nine patients completed the study as per protocol (DopApp™ n=20; placebo n=19). The mean age of participants was 67.1±6.7 years, 56% were male and PD duration was 5.64±3.1 years. Participant characteristics are shown in Table 1, with no significant between-group baseline differences. Neuroimaging was offered to all participants. Seven were ineligible, three withdrew the neuroimaging consent after the first scan, and one was excluded for excessive, in-test, motion artifact. With no group differences in head motion, the neuroimaging sub-study included 31 participants (DopApp™ n=17; placebo n=14). Eight participants experienced an adverse event (AE) (DopApp™ n=4; placebo n=4), all of which were considered unrelated to the treatment protocol of DopApp™/placebo. One serious AE (dyspnea requiring hospitalization) was observed in the placebo group.

TABLE 1

| | DopApp ™ | Placebo | Total |
|---|---|---|---|
| | (N = 20) | (N = 19) | (N = 39) |
| Age, yrs | 67.25 (6.7) | 66.9 (7.0) | 67.1 (6.7) |
| Male sex | 11 (55%) | 11 (58%) | 22 (56%) |
| PD duration, yrs | 5.85 (3.2) | 5.42 (3.2) | 5.64 (3.1) |
| Hoehn & Yahr Stage | | | |
| 0 | 0 (0%) | 0 (0%) | 0 (0%) |
| 1 | 8 (40%) | 5 (26%) | 13 (33%) |
| 2 | 11 (55%) | 14 (74%) | 25 (64%) |
| ≥3 | 1 (5%) | 0 (0%) | 1 (3%) |
| MDS-UPDRS total score | 36.55 (12.5) | 42.42 (18.6) | 39.41 (15.8) |
| Presence of motor fluctuations | 10 (50%) | 13 (68%) | 23 (59%) |
| Presence of dyskinesia | 7 (35%) | 6 (32%) | 13 (33%) |
| Duration of levodopa treatment, yrs/mos | 3.1 (1.5) | 2.8 (2.0) | 2.9 (1.8) |
| Daily levodopa dose, mg (range) | 526 (150-1460) | 500 (250-1100) | 513 (150-1460) |
| LEDD (range) | 626 (300-1560) | 692 (250-1475) | 659 (250-1560) |
| Antiparkinsonian medication use | | | |
| Dopamine agonists | 5 (21%) | 6 (31%) | 11 (28%) |
| MAO-B inhibitors | 11 (55%) | 15 (79%) | 26 (66%) |
| Amantadine | 7 (35%) | 6 (31%) | 13 (33%) |
| Exercise - Days per week | 5.4 (2.0) | 4.5 (2.0) | 4.9 (2.0) |
| Exercise - Minutes per day | 70.3 (39.5) | 64.5 (30.5) | 67.5 (35.1) |
| Questionnaires baseline | | | |
| MoCA | 27.15 (2.2) | 26.26 (1.8) | 26.72 (2.1) |
| PDQ39 | 19.84 (12.2) | 24.7 (15.2) | 22.21 (13.8) |
| BDI-II | 8.9 (7.1) | 9.16 (6.6) | 9.03 (6.8) |

### Adherence to Daily Treatment

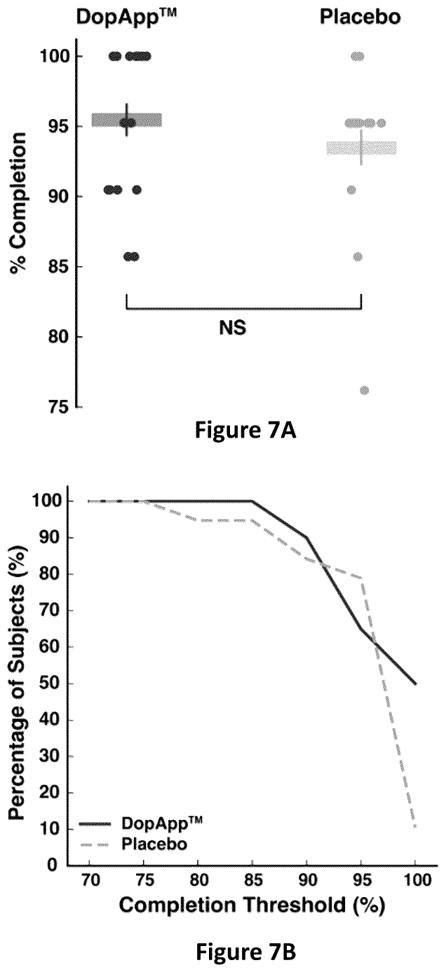

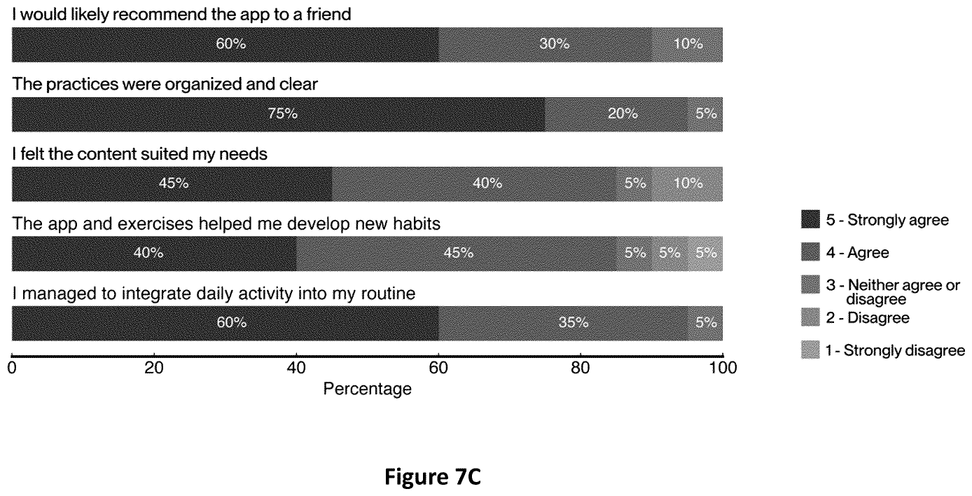

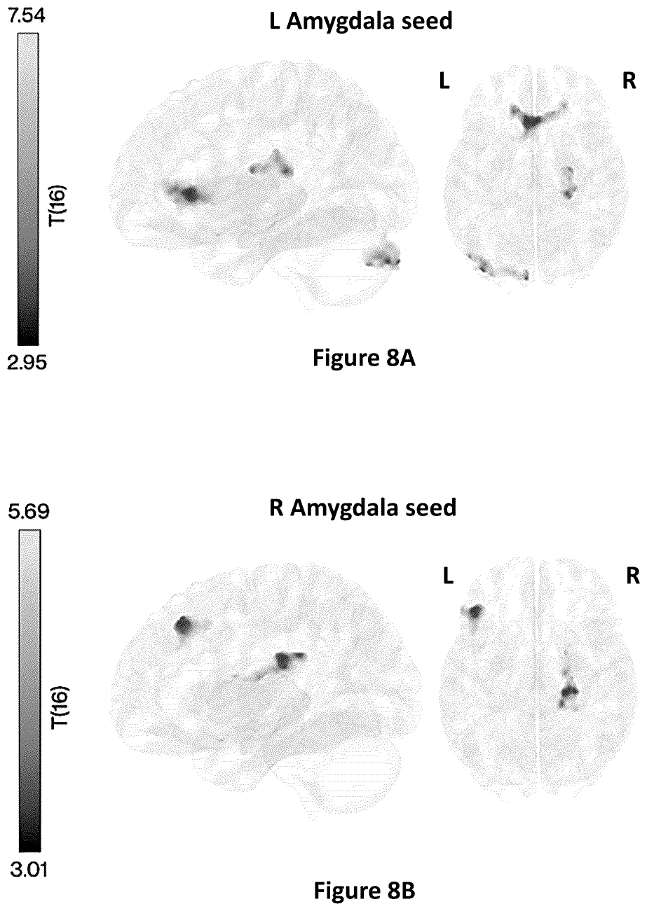

Daily treatment protocol adherence was high for both the DopApp™ and placebo groups, with completion rates of 95.5% and 93.5%, respectively, with no significant between-group differences (see FIGS. 7A-7C). In both groups, participants generally reported positive experiences with the app, particularly noting that it was easy to use and navigate. DopApp™ group participants reported a more positive experience when asked about whether the app met their expectations, contributed to their health, and their overall satisfaction (see FIGS. 7A-7C). The DopApp™ group also showed high ratings on additional app-specific questions, highlighting satisfaction with the clarity of practices, perceived usefulness of the content, successful integration into daily routines, and strong willingness to recommend the app to a fellow PD patient, with 90% indicating they would do so (see FIGS. 7A-7C).

Overall, 64.1% of participants correctly identified their group assignment (DopApp™ 55%, placebo 73.7%). When testing whether this distribution differed significantly from what would be expected by chance (50%), the result was not statistically significant (Chi-square test, p=0.078), suggesting that the blinding procedure was effective, with only a marginal indication of unblinding. Additionally, there were no significant differences in guessing accuracy between the groups (p=0.378), further supporting the blinding process' relative success.

### Clinical Outcomes

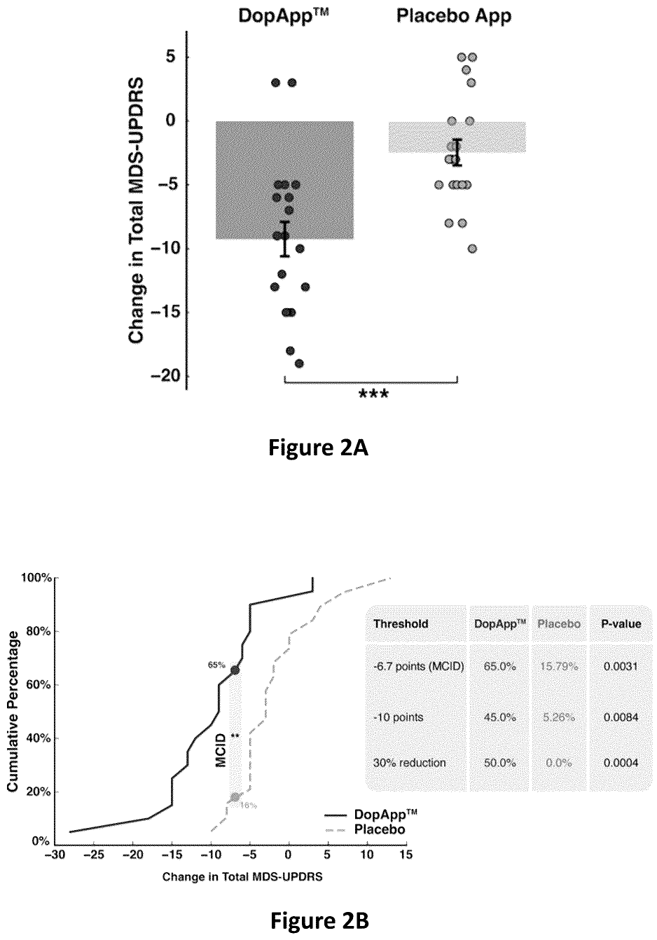

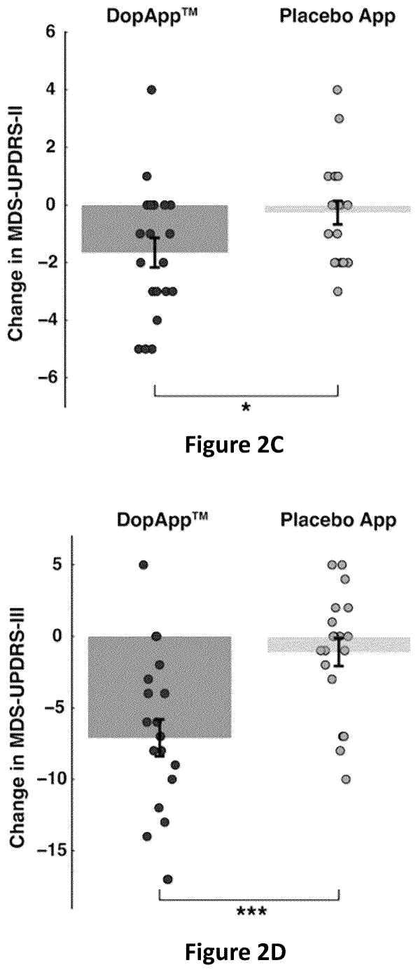

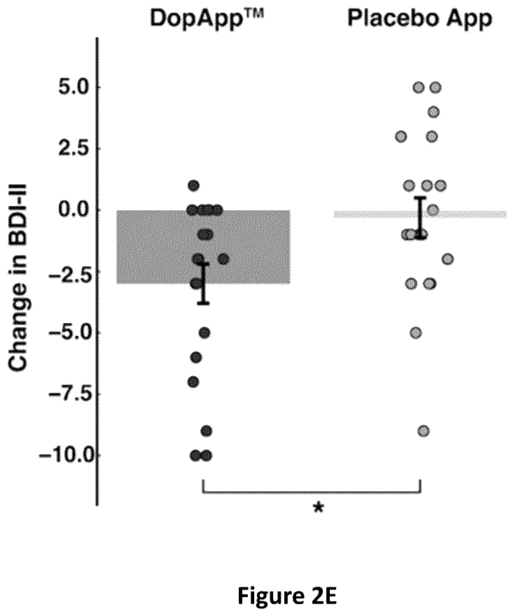

The study's primary therapeutic outcome was the change in MDS-UPDRS total scores (sum of parts I, II & III). Treatment with DopApp™ led to a significant improvement compared to placebo, with a mean±SD change from baseline in MDS-UPDRS total score of −9.7±7.03 in the DopApp™ group and −1.95±5.57 in the placebo group (P=0.0005, d=1.22, FIG. 2A). Likewise, a significantly greater response rate was observed in the DopApp™ group across all response thresholds, as illustrated by the distinct cumulative distributions of the two groups (FIG. 2B). Specifically, 65% of the DopApp™ group exceeded the minimal clinically important difference (MCID) of 6.7 points, whereas only 15.8% of the placebo group reached this threshold. Although the mean baseline MDS-UPDRS total score was slightly lower in the DopApp™ group, the difference was not statistically significant (P=0.25), and results remained unchanged when accounting for baseline variability. Further analysis of MDS-UPDRS part II (intergroup difference: 1.49 points; P=0.039, d=0.68) and MDS-UPDRS part III motor scores (intergroup difference: 6.46 points; P=0.0009, d=1.16) support the primary outcome (FIG. 2C, D). BDI-II depression scores improved by 3.5±5.02 in the DopApp™ group, compared to a negligible change of +0.05±4.31 in the placebo group, yielding a medium effect size (P=0.023, d=0.76) (FIG. 2E). All sensitivity analyses produced similar results with significant treatment differences. No significant intergroup differences were apparent for any other psychological, cognitive, or PD-related outcomes (see Table 2-Change from baseline in exploratory outcomes).

TABLE 2

| | | | | | | |
|---|---|---|---|---|---|---|
| | | Change from | | Change from | | |
| | Baseline | baseline | Baseline | baseline | Treatment | Effect |
| | DopApp | DopApp | Control | Control | difference | size |
| Variable | Mean ± SD | Mean ± SD | Mean ± SD | Mean ± SD | Mean, t(df) | Cohens |
| name | (N = 20) | (N = 20) | (N = 19) | (N = 19) | p value | D |

MDS-UPDRS assessments

| | | | | | | |
|---|---|---|---|---|---|---|
| Parts I, | 36.55 ± 12.49 | −9.7 ± 7.03 | 42.42 ± 18.58 | −1.95 ± 5.57 | 7.75, 3.8(37), | 1.22 |
| II & III | | | | | p = 0.0005 | |
| Parts I-IV | 39.95 ± 13.5 | −10.15 ± 7.5 | 47.11 ± 20.1 | −1.79 ± 6.8 | 8.36, 3.64(37), | 1.17 |
| | | | | | p = 0.0008 | |
| Part I | 5.5 ± 4.36 | −0.6 ± 3.39 | 8.16 ± 5.09 | −0.79 ± 3.07 | −0.19, −0.18(37), | −0.06 |
| | | | | | p = 0.8561 | |
| Part II | 6.85 ± 3.13 | −1.75 ± 2.49 | 8.05 ± 7.3 | −0.26 ± 1.79 | 1.49, 2.13(37), | 0.68 |
| | | | | | p = 0.0398 | |
| Part III | 24.2 ± 9.17 | −7.35 ± 6.29 | 26.21 ± 13.36 | −0.89 ± 4.68 | 6.46, 3.62(37), | 1.16 |
| | | | | | p = 0.0009 | |
| Part IV | 3.4 ± 3.52 | −0.45 ± 2.56 | 4.68 ± 3.65 | 0.16 ± 2.99 | 0.61, 0.68(37), | 0.22 |
| | | | | | p = 0.4988 | |
| Parts II & | 31.05 ± 10.85 | −9.1 ± 6.2 | 34.26 ± 16.9 | −1.16 ± 4.5 | 7.94, 4.56(37), | 1.46 |
| III | | | | | p = 0.0001 | |
| Parts I & | 12.35 ± 6.35 | −2.35 ± 5.3 | 16.21 ± 10.57 | −1.05 ± 3.87 | 1.3, 0.87(37), | 0.28 |
| III | | | | | p = 0.3904 | |
| Speech^{a} | 3.65 ± 2.41 | −0.45 ± 2.01 | 3.95 ± 2.22 | 0.16 ± 2.29 | 0.61, 0.88(37), | 0.28 |
| | | | | | p = 0.3838 | |
| Fine hand | 10.35 ± 3.84 | −3.1 ± 3.92 | 11.95 ± 6.62 | −0.21 ± 3.17 | 2.89, 37(37), | 0.81 |
| motor^{b} | | | | | p = 0.0161 | |

Questionnaires

| | | | | | | |
|---|---|---|---|---|---|---|
| PDQ-39 | 19.84 ± 12.29 | −6.46 ± 10.67 | 24.7 ± 15.21 | −3.42 ± 4.31 | 3.03, 1.15(37), | 0.37 |
| | | | | | p = 0.256 | |
| VHI-HEB | 16.1 ± 12.24 | −0.55 ± 12.08 | 21.74 ± 25.09 | 0.47 ± 7.27 | 1.02, 0.32(37), | 0.1 |
| | | | | | p = 0.752 | |
| PDSS-2 | 14.75 ± 8.96 | −2.7 ± 6.09 | 22.42 ± 12.5 | −4.21 ± 11.5 | −1.51, −0.52(37), | −0.17 |
| | | | | | p = 0.609 | |
| TMT-A | 48.25 ± 17.03 | −9.6 ± 10.46 | 50.63 ± 17.42 | −5.47 ± 10.6 | 4.13, 1.22(37), | 0.39 |
| | | | | | p = 0.229 | |
| TMT A + B | 145.35 ± 40.11 | −23.4 ± 31.46 | 182.42 ± 75.11 | −21.42 ± 32.94 | 1.98, 0.19(37), | 0.06 |
| | | | | | p = 0.849 | |
| BDI-II | 8.9 ± 7.14 | −3.5 ± 5.02 | 9.16 ± 6.59 | 0.05 ± 4.31 | 3.55, 2.36(37), | 0.76 |
| | | | | | p = 0.023 | |
| STAIT-5 | 6.85 ± 2.25 | −0.9 ± 1.55 | 7.68 ± 2.65 | −0.53 ± 1.78 | 0.37, 0.7(37), | 0.22 |
| | | | | | p = 0.488 | |
| STAIS-5 | 8.1 ± 2.34 | −1.1 ± 2.1 | 8.74 ± 3.31 | −0.47 ± 1.71 | 0.63, 1.02(37), | 0.33 |
| | | | | | p = 0.315 | |
| BRCS | 16.5 ± 3 | 0.25 ± 2.47 | 14.58 ± 4.39 | 2.11 ± 4.91 | 1.86, 1.5(37), | 0.48 |
| | | | | | p = 0.141 | |
| PSS-10 | 12.05 ± 5.59 | −3.35 ± 4.46 | 14.42 ± 5.55 | −1.95 ± 3.47 | 1.4, 1.09(37), | 0.35 |
| | | | | | p = 0.282 | |
| MHC-SF | 58.5 ± 13.22 | 4.5 ± 9.22 | 58.16 ± 11.03 | 2.11 ± 9.88 | −2.39, −0.78(37), | −0.25 |
| | | | | | p = 0.439 | |

^{a}MDS-UPDRS Speech (#2.1; #2.2; #2.3; #2.4; #3.1; #3.2),
^{b}MDS-UPDRS fine hand motor (#2.4; #2.5, #2.6, #2.7; #2.8, #3.4; #3.5, #3.6 #3.15, #3.16).
Abbreviations: PDSS-2, Parkinson's Disease Sleep Scale; PDQ-39, PD Questionnaire 39-item; VHI, Voice Handicap Index; PSS-10, Perceived Stress Scale; STAIT-5/STAIS-5, Brief State and Trait Anxiety Inventory; BDI-II, Beck's Depression Inventory II; MHC-SF, Mental Health Continuum Short Form; BRCS, Brief Resilient Coping Scale; TMT, Trail Making Test

### Motor Function Correlates: Clinical, Neural, and Digital Metrics

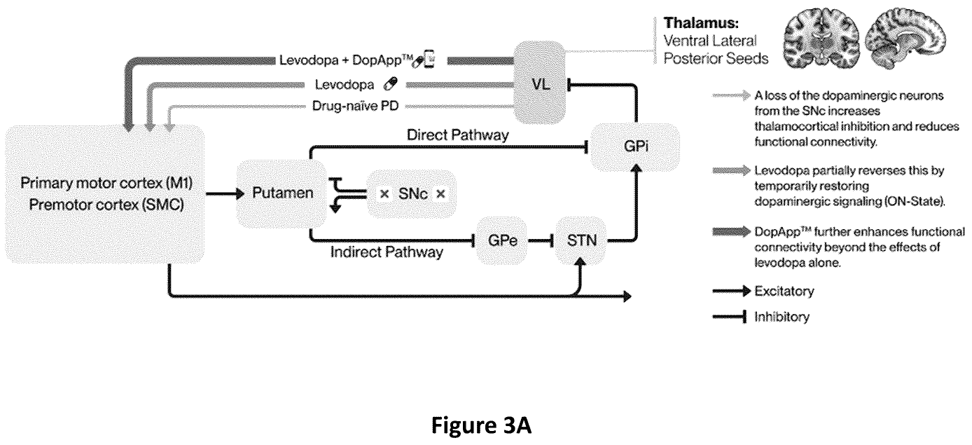

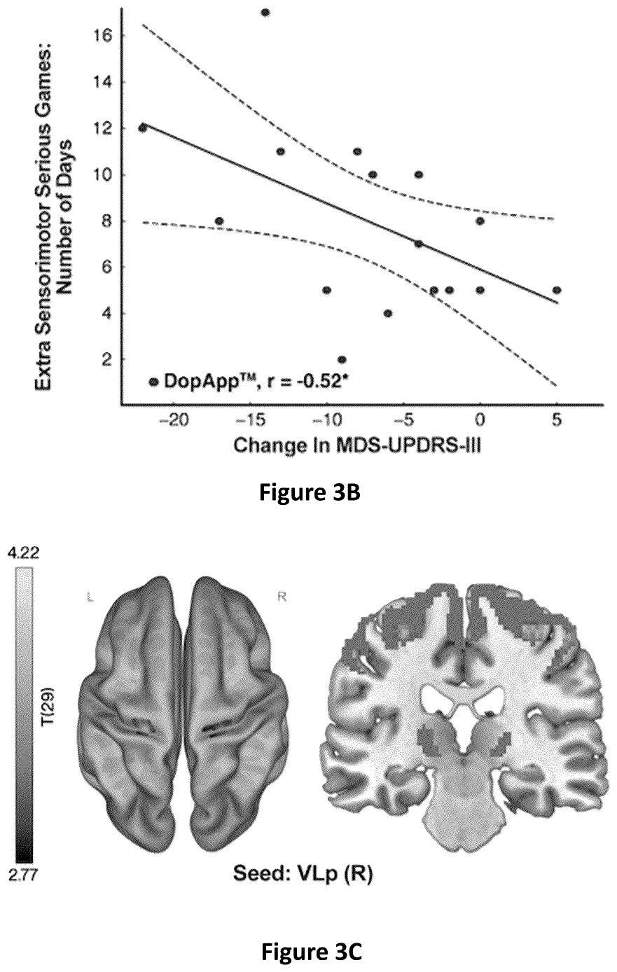

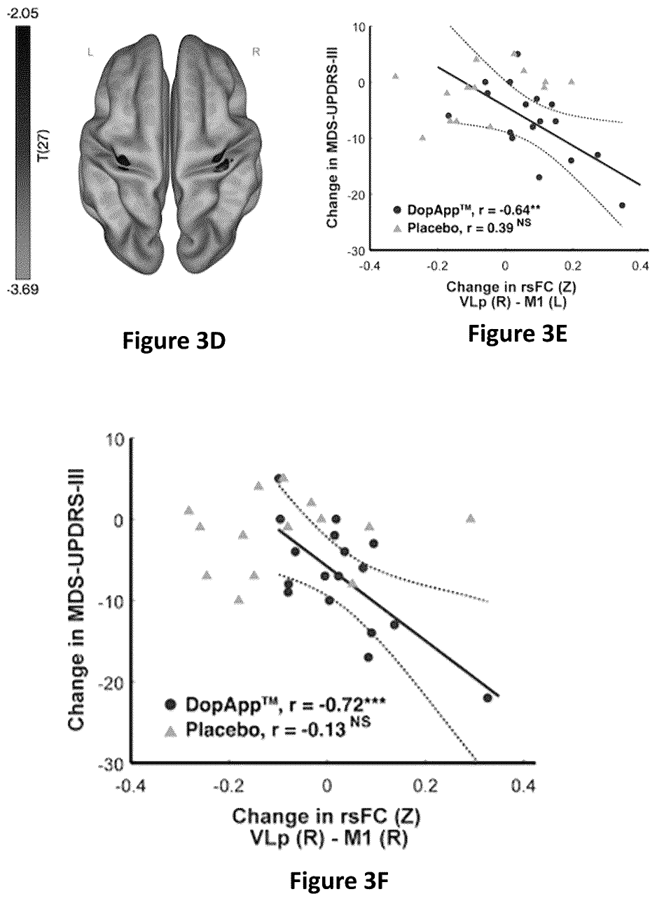

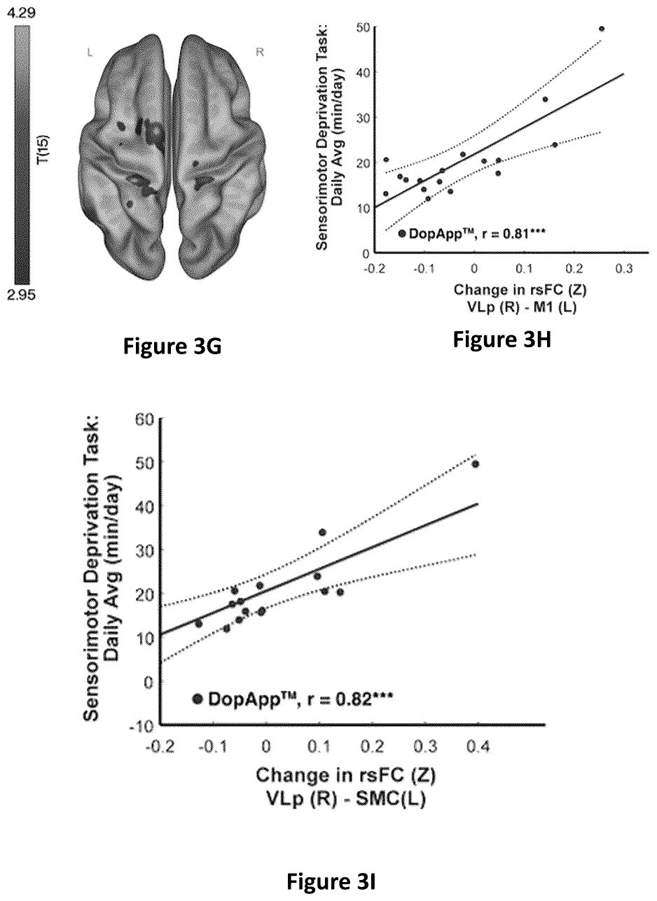

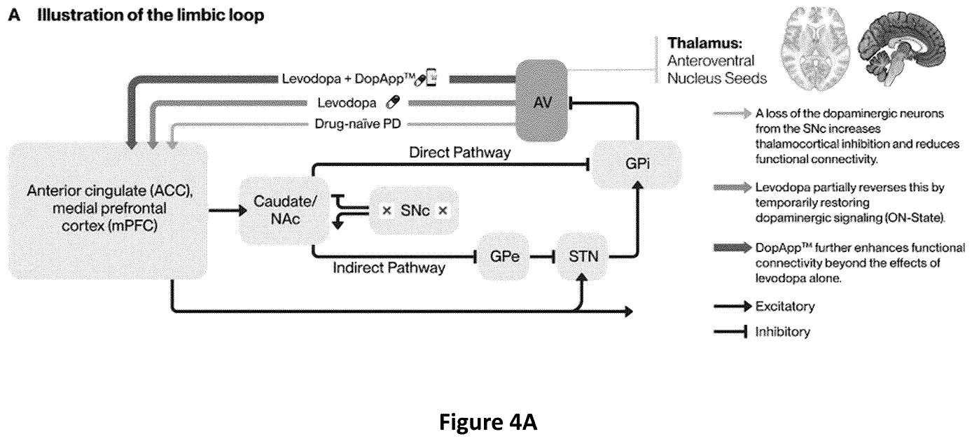

Motor outcomes were further examined through associations with complementary data: app engagement metrics, based on sensorimotor serious games data, and thalamocortical motor circuit alterations (FIG. 3A). Engagement metrics included exposure (total engagement time), daily-dose (average daily use), and usage consistency (number of active days). This multi-level approach revealed that improvements in MDS-UPDRS part III were significantly correlated with greater usage consistency in serious sensorimotor game sessions (r=−0.52, P<0.04; FIG. 3B), with no associations observed for emotion regulation components.

Next, it was found that these improvements at the clinical level were accompanied by functional reorganization within motor-related brain circuits. The hypothesis-driven ROI analysis was examined and focused on the motor ventral lateral (VL) thalamus nuclei and M1 to define significant areas for brain correlations. FIG. 3C shows a significant group-by-time interaction within the VL-M1 circuit, with increased rsFC between bilateral M1 clusters (Right: T=4.42, k=177; Left: T=4.01, k=100, P_{FDR}<0.05) and the right VLp seed. Additionally, a trend towards improved rsFC was also observed between the left VPL seed and bilateral M1 clusters (Right: T=3.39, k=36, Left: T=4.48, k=71, P_{FDR}<0.06). See Table 3 for more details. In line with the group-by-time results, increased rsFC between the right VLp and bilateral M1 were significantly correlated with reduced MDS-UPDRS part III scores (DopApp™: r=−0.72, −0.64, P<0.001, P<0.005, placebo: N.S, for the right and left M1 clusters respectively, FIGS. 3D-3F).

TABLE 3
Longitudinal changes in connectivity measures: Motor circuit.

| Seed- | Seed- | | | | | | | | | Cluster |
|---|---|---|---|---|---|---|---|---|---|---|
| HCPex | HCPex | Cluster | | | | | | Brain | | HCPex |
| ID* | area | (x, y, z) | k | T(29) | p-FWE | p-FDR | p-UNC | area | BA | ID* |
| 413 | VLp | +48 −8 +48 | 177 | 4.42 | <0.001 | <0.001 | <0.001 | M1 | 4 | 211 |
| | | −24 −22 60 | 100 | 4.01 | <0.001 | <0.001 | <0.001 | M1 | 4 | 31 |
| 381 | VPL | −18 −26 +62 | 71 | 4.48 | 0.033 | 0.059 | <0.001 | MI | 4 | 31 |
| | | +22 −26 +62 | 36 | 3.93 | 0.110 | 0.059 | <0.002 | M1 | 4 | 211 |
| 379 | VLa | +46 −12 +50 | 97 | 3.50 | 0.350 | 0.142 | <0.001 | M1 | 4 | 211 |
| 380 | VLa | NS | | | | | | | | |
| 414 | VPL | NS | | | | | | | | |
| 412 | VLa | NS | | | | | | | | |

*HCPex atlas, FWE, familywise error, FDR, false discovery rate, UNC, uncorrected, X, Y, Z MNI coordinates, BA, Brodmann area, VLa, ventral lateral anterior, VLp, ventral lateral posterior, VLP, ventral posterolateral motor related nuclei of the thalamus; M1, primary motor cortex, post > pre-intervention; DopApp > Placebo, p-value was analyzed at the peak-level within VL-M1 mask, k, cluster size, NS, not significant.

Exploring the associations between motor circuit alterations and app-based usage metrics yielded that the daily-dose of sensorimotor-deprivation tasks was strongly correlated with increased rsFC between the right VLp seed and both M1 (r=0.81, P<0.001) and supplementary motor cortex (SMC) (r=0.82; P<0.001; FIGS. 3G-3I) in the DopApp™ group. Taken together, these results suggest that repeated engagement with sensorimotor-deprivation components contributed to functional reorganization within thalamocortical motor circuits.

### Non-Motor Function Correlates: Clinical, Neural, and Digital Metrics

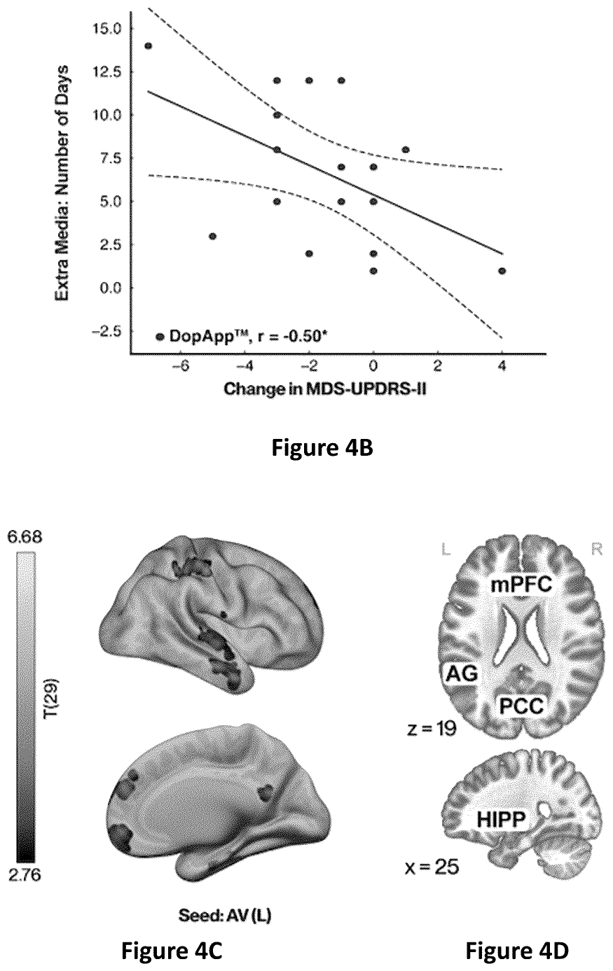

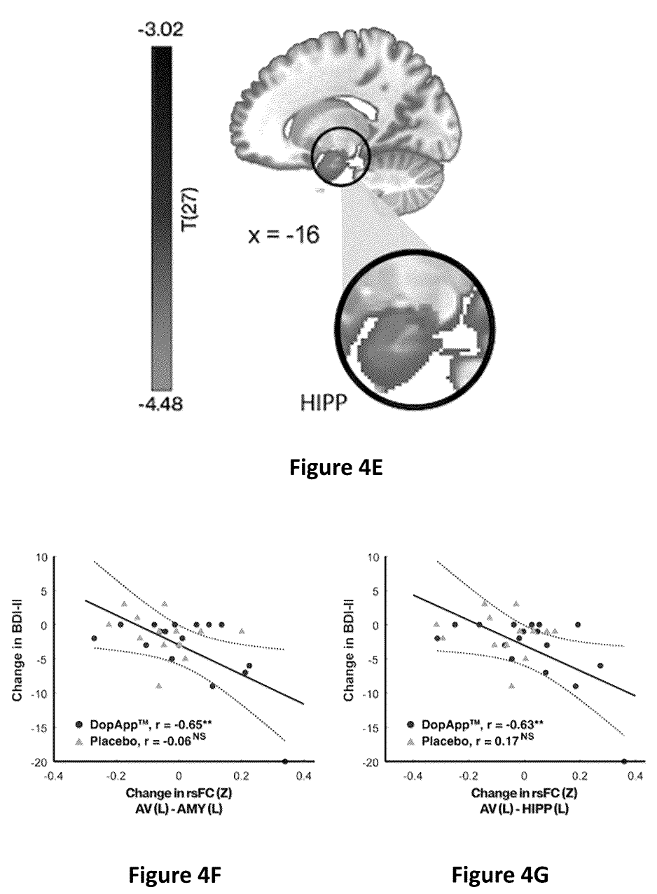

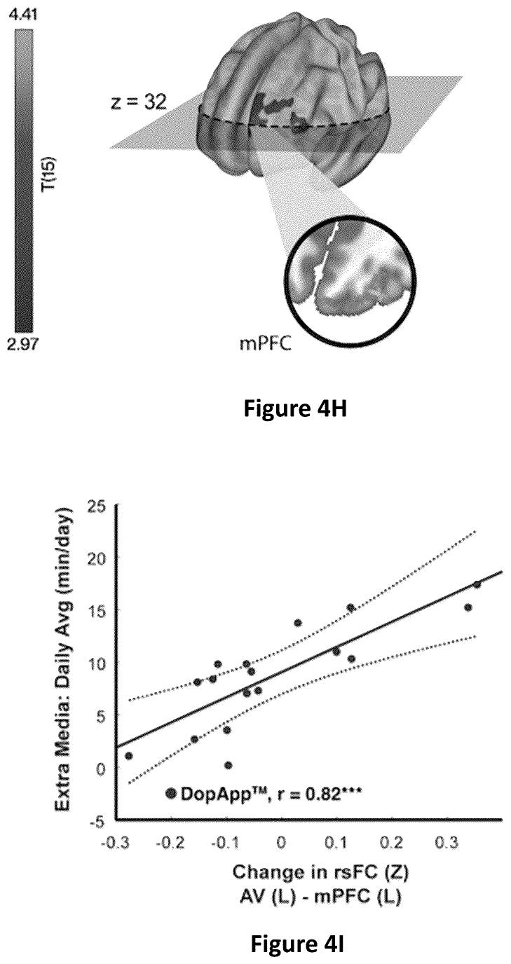

Similar to the above motor analysis, non-motor outcomes were further examined through associations with complementary data: engagement with app-based emotion regulation media content and thalamocortical limbic circuit alterations (FIG. 4A).

Analysis of app-based emotion regulation metrics revealed that usage consistency (number of engagement days) with this content significantly correlated with improvements in MDS-UPDRS Part II (r=0.50, P<0.05; FIG. 4B). Since Part II is indirectly yet substantially influenced by non-motor symptoms, this association suggests that sustained engagement contributed to the observed improvements.

The thalamocortical pathway was examined, focusing on thalamic subdivisions associated with the limbic network to delineate regions exhibiting significant connectivity changes to guide targeted correlation analyses. Following intervention, a significant group-by-time interaction was demonstrated, with increased rsFC between the left anteroventral (AV) thalamic nucleus with default mode network (DMN) nodes compared to placebo: mPFC (T=4.34, k=230, P_{FDR}<0.05), posterior cingulate cortex (PCC), (T=4.15, k=237, P_{FDR}<0.05), and left angular gyrus (AG) (T=4.19, k=346, P_{FDR}<0.05). Additionally, a group-by-time interaction within the medial temporal lobe (MTL)-AV pathway was observed, specifically in bilateral clusters localized in the hippocampus (T=4.36, 3.46, k=89, 215, P_{FDR}<0.05), and in the para-hippocampus (T=6.67, 2.91, k=313, 38, P_{FDR}<0.05) compared to placebo (FIGS. 4C-4D, Table 4).

TABLE 4
Longitudinal changes in connectivity measures: Limbic circuit

| Seed- | Seed- | Peak | | | | | | | | Cluster |
|---|---|---|---|---|---|---|---|---|---|---|
| HCPex | HCPex | Cluster | | | | | | Brain | | HCPex |
| ID* | area | (x, y, z) | k | T(29) | p-FWE | p-FDR | p-UNC | area | BA | ID* |
| 361 | AV | +56, +04, −36 | 650 | 4.45 | <0.001 | <0.001 | <0.001 | TG | 20 | 274 |
| | | +58, −10, −8 | 203 | | | | | Auditory5 | 22 | 241 |
| | | +56, −02, −24 | 196 | | | | | STS | 21 | 245 |
| | | +52, +06, −34 | 53 | | | | | TG | 38 | 274 |
| | | +62, −14, +6 | 51 | | | | | Auditory4 | 41 | 240 |
| | | +62, −10, −18 | 50 | | | | | TE | 21 | 269 |
| | | +50, −26, +52 | 555 | 4.25 | <0.001 | <0.001 | <0.001 | PSC | 1 | 208 |
| | | −54, −66, +16 | 346 | 4.19 | 0.004 | 0.001 | <0.001 | LOC | 39 | 99 |
| | | +28, −20, −24 | 313 | 6.67 | 0.008 | 0.002 | <0.001 | ParaH | 36 | 262 |
| | | +26, −16, −20 | 89 | 4.36 | | | | Hippocampus | | 260 |
| | | +10, +54, +24 | 255 | 4.61 | 0.027 | 0.005 | <0.001 | dlPFC | 9 | 319 |
| | | −12, −52, +18 | 237 | 4.15 | 0.039 | 0.007 | <0.001 | PCC | 23 | 129 |
| | | +02, +60, −10 | 230 | 4.34 | 0.046 | 0.007 | <0.001 | mPFC | 10 | 315 |
| | | −22, −12, −24 | 215 | 3.46 | 0.063 | 0.008 | <0.001 | Hippocampus | | 80 |
| 394 | AV | +02, +58, −20 | 202 | 3.87 | 0.087 | 0.056 | 0.001 | mPFC | 10 | 315 |
| 369 | MD1 | NS | | | | | | | | |
| 402 | MD1 | −32, −74, +14 | 409 | 5.12 | 0.001 | <0.001 | <0.001 | LOC | 19 | 25 |
| | | −40, +02, +42 | 210 | 4.14 | 0.062 | 0.026 | <0.001 | PreMot | 6 | 47 |
| 370 | MDm | −38, −68, +08 | 305 | 4.59 | 0.010 | 0.005 | <0.001 | MT(V5) | 19 | 23 |
| | | −44, −68, +8 | 132 | | | | | LOC | 19 | 18 |
| 403 | MDm | −42, +00, +38 | 224 | 4.21 | 0.058 | 0.039 | <0.001 | PreMot | 6 | 47 |
| 387 | Amyg | NS | | | | | | | | |
| 420 | Amyg | NS | | | | | | | | |

*HCPex atlas, FWE, familywise error, FDR, false discovery rate, UNC, uncorrected, X, Y, Z MNI coordinates, BA, Brodmann area, AV, anteroventral , MDI, mediodorsolateral parvocellular, MDm, mediodorsomedial magnocellular limbic-related nuclei of the thalamus; TG, temporal gyrus, STS, superior temporal sulcus, PSC, primary sensory cortex, TE, inferior temporal cortex, LOC, lateral occipital cortex, ParaH, parahippocampal gyrus, dlPFC, dorsolateral prefrontal cortex, mPFC, medial prefrontal cortex, PreMot, premotor cortex, MT, medial temporal, whole-brain, Post > Pre-intervention; DopApp > Placebo, k, cluster size, NS, not significant.

In line with the group-by-time results, it was also found significant correlations between reductions in BDI-II depression scores and increased rsFC between the left AV nucleus and both the left amygdala and left hippocampus (r=−0.65, P<0.005; r=−0.63, P<0.007 respectively, placebo: N.S, FIGS. 4E-4G). Regarding app usage, increased rsFC between the left AV and the medial prefrontal cortex (mPFC; BA9, 10) was correlated with higher self-initiated daily doses of emotion regulation content usage (r=0.82, p<0.001, FIGS. 4H-4I). This neural pathway, which is part of the DMN, is implicated in self-referential processing and emotional regulation. Importantly, no significant relationship was found between changes in VL, the motor thalamic nuclei, connectivity, and emotion regulation activity measures, supporting the hypothesis of distinct and parallel neural pathways underlying the intervention's motor and emotional components.

### Amygdala Network Correlates: Clinical, Neural, and Digital Metrics

Testing the amygdala seeds, no group-by-time interaction effects were observed. Nevertheless, significantly increased post-intervention connectivity was found in the DopApp™ group between the left amygdala and the left caudate, and between the right amygdala and the right thalamus (P_{FDR}<0.05). Importantly, significant associations were found between rsFC and both psychological state measures and related app engagement measures. In the DopApp™ group, increased rsFC within the right amygdala-AV pathway was correlated with improvements in both BDI-II (r=−0.75, P<0.001) and MDS-UPDRS Part II (r=−0.66, P<0.005) scores, while no significant correlations were found in the placebo group (FIGS. 5A-5B). Notably, a significant correlation was also observed between changes in BDI-II and MDS-UPDRS Part II scores (r=0.62, P<0.008; FIG. 5C). These findings, along with the involvement of the AV, as a limbic subregion of the thalamus, further support the interpretation that the MDS-UPDRS Part II measure is influenced by emotional NMS in PD.

The right amygdala network was also implicated in motivational processes, as illustrated in FIG. 5D. To quantify this, the self-initiated sensorimotor serious games measures were analysed, which reflect involvement in cognitively demanding and reward-based tasks. It was found that the higher daily-dose of this measure was correlated with increased rsFC between the right amygdala and the orbitofrontal cortex (BA 11) (r=0.72, P<0.001), in contrast to the rsFC patterns observed with self-initiation with emotion regulation activities (FIG. 5D). The amygdala-orbitofrontal pathway is well established in supporting motivation and reward-based decision-making.⁷⁵ Notably, self-initiated daily-doses of the sensorimotor-deprivation task, the most demanding of the reward-based components, was specifically correlated with improvements in MDS-UPDRS Part II scores, suggesting a link between self-motivation and improvements in subjective perception of motor function (r=−0.5, P<0.04; FIG. 5E).

Exploring associations within the left amygdala network revealed significant correlation between increased rsFC in the left amygdala-caudate pathway and the total exposure to emotion regulation content (r=0.72, P<0.001; FIG. 5F). Interestingly, this usage measure was also correlated with improvements in MDS-UPDRS Part-II scores (r=−0.58, P<0.02; FIG. 5G). Additionally, it was found that increased rsFC in the left amygdala-insula pathway was correlated with the extent of BDI-II score reductions (r=−0.66, P<0.005; FIG. 5H).

### DISCUSSION

In this study, it was demonstrated that a three-week use of a multimodal digital intervention (DopApp™) used alongside standard levodopa therapy significantly improved motor and non-motor outcomes in PwP. These effects were associated with parallel modulation of rsFC within the thalamocortical motor and limbic networks. Moreover, increased engagement with sensorimotor and emotion regulation activities was associated with both enhanced therapeutic effects and rsFC alterations, suggesting a digital dose-response relationship and task-specific neural plasticity. These results provide preliminary evidence that digitally delivered, behaviorally targeted interventions can augment dopaminergic pharmacotherapy benefits by promoting brain functional reorganization and may offer a scalable method to extend established PD treatment efficacies.

Consistent with the first hypothesis, DopApp™ led to both statistically significant and clinically meaningful improvements in MDS-UPDRS total scores among levodopa-treated PwP, primarily driven by changes in Part III: Motor Examination. The magnitude of improvement was notable for a three-week digital intervention. Several factors likely contributed to these rapid and robust therapeutic effects. DopApp™'s multidimensional design, targeting cognitive, emotional, sensorimotor, and motor domains, aligns with clinical guidelines for comprehensive PD care. The app also utilizes sensorimotor-deprivation and neuroscience elements, which enhances neuroplasticity and brief, gamified tasks, helped sustain user engagement and high adherence. This shows that digital intervention may complement standard dopaminergic treatment by augmenting therapeutic effects across motor and NMS.

In addition to the motor improvements, it was also observed that improvements in both BDI-II depression scores and MDS-UPDRS Part II (FIGS. 2A-2E). The reduction in depression, a key NMS of PD, aligns with prior evidence showing that digital non-pharmacological interventions can improve mood in PwP. Although MDS-UPDRS Part II primarily assesses motor experiences of daily living, it is strongly influenced by non-motor factors such as mood, motivation, and emotional wellbeing, reinforcing its relevance for NMS evaluation. Notably, these effects were achieved without therapist involvement, relying entirely on smartphone-based self-engagement, distinguishing this intervention from prior approaches.

Consistent with the second hypothesis, the intervention modulated motor thalamocortical connectivity. Engagement with sensorimotor-deprivation modules that utilize multisensory spatial memory tasks, was associated with reduced MDS-UPDRS Part III scores and with corresponding connectivity alterations (FIG. 3A). Thalamocortical motor connectivity in PD and improvements follow therapy. Given that spatial learning deficits in PD relate to both motor and non-motor symptoms, the observed effects may reflect the combined impact of targeted training on motor function and broader network integration. Additionally, while thalamocortical projections are predominantly ipsilateral, it was observed that enhanced bilateral connectivity in the DopApp™ group compared to placebo. Several mechanisms may account for this. Firstly, the cerebello-thalamocortical loop is double-crossed, creating apparent contralateral pathways. Secondly, interhemispheric synchrony via the corpus callosum maintains phase-locked M1 regions. Thirdly, a small subset of VL thalamic neurons project contralaterally. These mechanisms may reflect compensatory processes reconciling ipsilateral wiring with strong contralateral motor correlations, consistent with neuroplastic compensatory patterns seen in stroke and aging.

Regarding the third hypothesis, it was observed that widespread connectivity increases between the AV thalamic nucleus and limbic and DMN regions, including the hippocampus, para-hippocampus, mPFC, PCC and AG (see FIGS. 4A-4I). The AV nucleus, a core node of the hippocampal-anterior thalamic axis, is critically involved in memory and emotional regulation, and is implicated in PD-related apathy, anxiety, and cognitive decline. Functional disruptions of this nucleus have been reported in both PD and Alzheimer's disease, contributing to deficits in memory and emotion. In the present study, enhanced AV-DMN coupling suggests improved integration within limbic processing circuits, and increased coupling with MTL structures, including bilateral hippocampi and para-hippocampi, further supporting the AV's role in modulating memory and affective function. Furthermore, greater exposure to psychological content was correlated with improvements in emotional and self-perceived motor function, alongside altered limbic-DMN connectivity. The absence of such associations with motor thalamic (VL) connectivity supports these effects' circuit-specificity.

A significant group-by-time interaction involving the amygdala was not observed. However, increased connectivity within the amygdala-striatum-thalamus pathway was observed in the DopApp™ group. Notably, this increase was significantly correlated with improvements in BDI-II and MDS-UPDRS Part II scores in the DopApp™ group, but not in the placebo group (FIGS. 5A-5H). These results suggest meaningful individual-level effects, despite low baseline psychopathology levels in this cohort.

Quantifying the efficacy of digital interventions remains a major challenge, and identifying dose-response relationships is critical for establishing therapeutic validity and optimizing treatment protocols. Here, the potential dose-response dynamics using app engagement metrics was examined, including overall exposure, daily-dose, and usage consistency. These metrics reflect real-world usage and help identify effective engagement thresholds. For instance, increased exposure to psychological content beyond a certain time threshold was associated with enhanced rsFC, suggesting a usage efficacy threshold. Moreover, participants' preferences for specific activity types (e.g., reward-based vs. relaxation-focused) were linked to distinct patterns of rsFC alterations. These findings indicate that different content types may require different effective dosages to drive neural changes, highlighting the need for personalized therapeutic pathways. Overall, this underscores the importance of targeting and tailoring digital intervention components, both in type and dose, to specific clinical and neural outcomes.

Another important aspect of this study is its placebo-controlled design, addressing the unique challenge of developing a credible digital placebo that replicates engagement without therapeutic elements. Although blinding held statistically, results approached the conventional threshold, suggesting a potential marginal unblinding effect. Still, improvements observed in the placebo arm are consistent with a placebo effect, further supporting effective blinding. Future investigations should aim to optimize placebo design to minimize unblinding risk. Additionally, including a heterogeneous cohort spanning a broad range of disease severities and ages enhances the findings' ecological validity and generalizability. Reliance on widely available standard smartphones, including among older adults, eliminates the need for specialized hardware, thereby enhancing the intervention's scalability and clinical feasibility, particularly in resource-constrained settings. Importantly, despite a mean age of 67 and the presence of motor impairments, adherence rates were high, indicating that neither digital literacy nor physical limitations posed substantial barriers in this population. Finally, as a non-pharmacological intervention, DopApp™ is devoid of drug-related adverse effects, offering a favorable safety profile for individuals with PD.

In conclusion, the study provides preliminary clinical and neurobiological evidence that a targeted, digital therapeutic can augment standard dopaminergic treatment in PD. By engaging multidimensional digital activities, DopApp™ improved motor function, mood, and daily living, while modulating thalamocortical connectivity in motor and limbic circuits. Dose-response relationships between engagement and outcomes suggest that adaptive, task-specific digital tools may drive circuit-selective plasticity. These findings support the potential of scalable, adjunctive digital interventions and their integration into precision, drug-digital treatment strategies for neurodegenerative diseases. Furthermore, they represent an early step toward the development of hybrid digital-drug interventions that may personalize care, enhance pharmacological efficacy, and extend therapeutic reach beyond the traditional clinical setting.

### Example 3

### Personalization Framework

A personalization framework may be implemented to tailor therapeutic interventions based on a wide range of individual-specific parameters. Inputs to the personalization algorithm include clinical, behavioral, physiological, and preference-based data.

Clinical inputs may include the individual's disease stage, such as classification using the Hoehn & Yahr staging system, Movement Disorder Society Unified Parkinson's Disease Rating Scale (MDS-UPDRS) staging, or combinations thereof, and detailed disease characteristics. These characteristics encompass both motor symptoms, such as gait disturbances, balance issues, dexterity impairments, and tremors, and non-motor symptoms, including cognitive slowing, fatigue, mood fluctuations, and sleep disturbances.

Behavioral and physiological inputs may include physical activity logs detailing activities performed outside the application. These logs may capture workout type, duration, timing, energy expenditure, distance, and workout-specific metrics such as stroke count or running power. Physiological metrics may include heart rate, walking heart rate average, and estimated VO₂Max.

Personal profile and preference data are also used to guide personalization. Examples include age, hobbies (e.g., a patient who enjoys wildlife may receive nature-themed content), preferred music, spoken language, caregiver involvement preferences, educational background, and professional history. The framework may also incorporate patient-defined behavioral goals to align therapeutic content with individual motivations.

Treatment-related data may include drug name, dosage, timing, form (e.g., capsule, tablet, injection), and intake tracking, such as binary indicators of whether the drug was taken and the corresponding date and time. On-Off states, whether self-reported or measured by the application, are also considered.

Performance metrics collected within the application further inform personalization. These may include results from standardized tasks such as the Timed Up and Go (TUG) test, which measures functional mobility by timing how long it takes a person to rise from a chair, walk a short distance, turn, return, and sit down. Additional metrics may include typing speed, voice amplitude, and performance on cognitive tasks such as digital mazes.

Input data is collected from multiple sources, including personal health information provided during onboarding or through updates, patient-reported outcomes via questionnaires, interaction data generated within the application, and data collected from wearable devices.

Personalization affects several aspects of the therapeutic experience, including the selection of modules and types of interventions based on changing needs, tailoring of content to individual preferences to enhance engagement and motivation, and dynamic adjustment of sequencing and frequency of interventions to optimize therapeutic outcomes.

### Personalization for Patient X

Patient X is a 58-year-old individual diagnosed with Parkinson's disease, currently classified as Hoehn & Yahr Stage 2. This stage indicates mild bilateral involvement with minimal balance impairment. Her motor symptoms include a mild hand tremor and reduced dexterity, particularly noticeable when performing tasks such as buttoning clothes. Non-motor symptoms are present but relatively mild, consisting of occasional fatigue and sleep disturbances-specifically, waking once per night.

Outside the therapeutic application, Patient X maintains a modest level of physical activity. She takes a 20-minute walk with her husband after dinner and engages in gardening twice per week. However, she does not follow a formal exercise routine. Physiological data collected via wearable devices show a resting heart rate of 70 bpm, an average walking heart rate of 88 bpm, and a VO₂Max of 31 ml/kg/min, which is typical for her age group.

Her personal profile and preferences provide further insight into her lifestyle and therapeutic needs. She is a native Spanish speaker who enjoys gardening and playing classical guitar. Her preferred music includes Spanish guitar and instrumental compositions. She lives independently and reports minimal caregiver involvement. Her self-defined behavioral goals focus on improving hand coordination and enhancing sleep quality.

Patient X is currently prescribed Levodopa/Carbidopa 25/100 mg tablets, taken three times daily at 7:30 AM, 12:30 PM, and 6:30 PM. Her medication adherence is excellent, with 100% compliance logged via the app. She experiences stable On-Off states, with mild “wearing-off” symptoms occurring near the end of each dosing interval.

In-app performance metrics further support her clinical profile. She completes the Timed Up and Go (TUG) test in 8.4 seconds, which falls within the normal range. Her typing speed is stable at 32 words per minute, and her voice amplitude averages 67 dB, also within normal limits. On cognitive tasks such as digital mazes, she demonstrates high accuracy, scoring 95%.

Patient X's therapeutic plan is personalized to address her primary concerns: fine motor control and sleep quality. Given her moderate physical stamina, the application prescribes one focused training session per day. These sessions are strategically balanced throughout the week to alternate between cognitive and physical exercises, ensuring a manageable pace that supports engagement without inducing fatigue.

The system employs a dynamic adaptation mechanism to refine Patient X's weekly plan based on real-time data. If her fatigue scores increase, whether reported directly or inferred from wearable data, cognitive tasks are prioritized earlier in the day when her energy levels are typically higher. As her hand dexterity improves, the difficulty of coordination tasks is progressively increased by introducing smaller interaction targets and faster timing requirements. When sleep quality improves, the frequency of relaxation modules is reduced, and gait training sessions may be introduced midweek to further support motor function.

The outcomes of this personalization strategy are multifaceted. The content of therapeutic tasks is tailored to reflect Patient X's interests, incorporating gardening themes and Spanish guitar music to enhance relevance and enjoyment. Engagement is supported by limiting the plan to one clear, manageable task per day, minimizing the risk of fatigue or frustration. Clinically, the tasks are aligned with her mild disease stage and stable motor function, ensuring appropriate challenge without overexertion. Finally, the system leverages longitudinal data from wearable devices and in-app performance metrics to guide weekly adjustments, maintaining a responsive and individualized therapeutic experience.

1. A method for modulating one or more Parkinson's disease-related markers, symptoms, or disease progression in an individual in need thereof, the method comprising:
determining a therapeutic plan to be delivered to the individual, wherein the therapeutic plan comprises therapeutic interventions selected from: at least one motor skills intervention, at least one psychological intervention, at least one cognitive intervention, or a combination thereof;
converting the therapeutic plan into a plurality of targeted interaction outputs for presentation to the individual; and
adaptively delivering the therapeutic plan, tracking the individual's responses and associated performance metrics, and continuing until a predetermined condition is met.

2. The method of claim 1, further comprising obtaining a plurality of attributes associated with the individual from a plurality of data acquisition sources to generate an initial profile for the individual, wherein the determining of the therapeutic plan further comprises adapting the therapeutic plan based on the initial profile.

3. The method of claim 1 wherein the therapeutic plan comprises performing the at least one motor skill intervention concurrently with the at least one cognitive intervention or the at least one psychological intervention.

4. The method of claim 2, wherein the adapting of the therapeutic plan further comprises selecting and implementing therapeutic intervention parameters based on at least one attribute of the individual selected from: age, hobbies, music preferences, spoken language, caregiver involvement preferences, disease characteristics, disease severity, symptoms, educational background, professional background, and individual-defined goals.

5. The method of claim 1 further comprising administering at least one medicinal agent before, during, after, or a combination thereof, the implementation of at least one of the therapeutic interventions.

6. The method of claim 5 wherein the at least one medicinal agent comprises a dopamine precursor and optionally in combination with an adjunct agent.

7. The method of claim 6 wherein the dopamine precursor and optionally in combination with an adjunct agent comprises any of the following selected from: levodopa, levodopa/carbidopa, levodopa/benserazide, or combinations thereof.

8. The method of claim 5 wherein the therapeutic plan is further adapted to the dopaminergic state of the individual selected from an ON state or an OFF state, wherein the selection, intensity, and scheduling of the therapeutic interventions is optimized based on whether the individual is in the ON state or the OFF state.

9. The method of claim 5 wherein the at least one medicinal agent comprises any of the following selected from: dopaminergic agents, dopamine precursors, dopamine agonists, COMT inhibitors, MAO-B inhibitors, peripheral DOPA decarboxylase inhibitors, glutamatergic modulators, anticholinergic agents, antidepressant agents, anxiolytic agents, antipsychotic agents, cognitive enhancing agents, alpha-synuclein targeting agents, LRRK2 inhibitors, adenosine A2A receptor antagonists, GLP-1 receptor agonists, incretin-based therapies, gene therapy agents, viral-vector-mediated gene therapies, neurotrophic factor modulators, sigma-1 receptor modulators, muscarinic receptor modulators, neuroprotective or neuronal-homeostasis-restoring agents, inflammasome modulators, neuro-inflammation modulators, immunomodulators, immunosuppressants, G-protein-coupled receptor modulators, GCase modulators, mucolytic agents, anticonvulsants, beta-blockers, vitamins, cofactor supplements, cannabinoids, symptom-managing adjunct agents, cell-based dopaminergic neuron replacement therapies, and stem-cell-derived dopaminergic neuron replacement therapies, D1/D5 dopamine receptor partial agonists, tavapadon, prasinezumab, BIIB122, DNL151, lixisenatide, exenatide, blarcamesine, NLRP3 inhibitors, GPR6 inverse agonists, and solengepras, or combinations thereof.

10. The method of claim 1 further comprising administering light therapy before, during, after, or a combination thereof, at least one of the therapeutic interventions.

11. The method of claim 2 further comprising:
dynamically monitoring the individual's monitored parameters during delivery of the therapeutic plan;
generating a dynamic user profile based on the individual's monitored parameters selected from: the initial profile, Electronic Medical Records (EMR) data, motor attributes, psychological attributes, cognitive attributes, pharmacological attributes, feedback of the individual, feedback of a medical practitioner, drug intake parameters, or a combination thereof; and
adapting the therapeutic plan based on the dynamic user profile.

12. The method of claim 11 further comprising recursively adapting the therapeutic plan based on updates to the dynamic user profile, the performance metrics, and the individual's responses.

13. The method of claim 12, wherein the recursive adapting of the therapeutic plan comprises dynamically identifying and modifying intervention parameters to implement optimized therapeutic outcomes towards a predetermined therapeutic goal.

14. The method of claim 13 wherein the intervention parameters comprise any of the following selected from: type, timing, dosage, intensity, sequence, duration, frequency, mode of delivery, level of guidance, feedback modality, language adaptation, complexity level, interface configuration, integration with external systems, or a combination thereof, of one or more of the therapeutic interventions.

15. The method of claim 12 wherein the recursive adapting is performed using a feedback control algorithm, Bayesian inference, or a combination thereof.

16. The method of claim 1 wherein the at least one motor skills intervention is selected from: speech and language therapy, physiotherapy, tremor management, mobility training, freezing and rigidity exercises, tapping training, limb agility exercises, training for freezing of gait, motor function therapy, dance therapy, handwriting training, balance exercises, postural stability training, strength training, stretching exercises, coordination training, metronome training, Tai-chi, physical exercises, walking, dual tasking exercises, and fine motor skills training, or a combination thereof.

17. The method of claim 1 wherein the at least one psychological intervention is selected from: guided imagery, psychoeducation, psychotherapy, cognitive behavioral therapy, stress and anxiety management training, mindfulness-based interventions, body scanning training, sleep hygiene, fatigue training, acceptance and commitment therapy (ACT), dialectical behavior therapy (DBT), attention training techniques, psychodynamic therapy, solution-focused brief therapy (SFBT), narrative therapy, pain therapy, addiction therapy, gestalt therapy, behavioral activation therapy, adjustment therapy, grief therapy, motivational therapy, meaning-centered therapy, creative therapy, expressive therapy, logotherapy, telepsychiatry, and teletherapy, or a combination thereof.

18. The method of claim 1 wherein the at least one cognitive intervention is selected from: navigation in a maze, navigation in a digital maze, spatial navigation, working memory training, magic-7 training, memory enhancement techniques, executive function training including, problem-solving training, planning training, cognitive flexibility training, inhibition training, attention training, concentration training, processing speed training, sonification exercise, data visualization, geometric puzzles, shape and pattern matching, visual perception tasks, spatial reasoning games, face detection training, drawing game, focus training, reading training, cueing training, metacognition training, memory retrieval training, dual tasking training, or any combination thereof.

19. The method of claim 1 wherein each of the therapeutic interventions has a duration ranging between 10s to 60 mins.

20. The method of claim 1 wherein the predetermined condition is selected from: a predetermined number of iterations, a predetermined duration has elapsed, a predetermined number of therapeutic interventions have been completed, predetermined progress measurement, predetermined regression measurement, target threshold score achieved, transition to maintenance therapy, and no further improvement is measured, or a combination thereof.

21. The method of claim 1 wherein the targeted interaction outputs are selected from: visual, auditory, tactile, or a combination thereof.

22. The method of claim 21 wherein at least one of the therapeutic interventions further comprises: sensory inhibition, sensory substitution, sensory integration, or a combination thereof.

23. The method of claim 5 further comprising a conditioning stimulus delivered before, during, after, or a combination thereof, the administering of the at least one medicinal agent, at least one of the therapeutic interventions, or a combination thereof.

24. The method of claim 1 wherein the Parkinson's disease-related markers comprise any of the following selected from: Unified Parkinson's Disease Rating Scale (UPDRS) score, motor symptoms, tremor amplitude, gait symmetry, or reaction-time variability, cognitive function, autonomic function, fatigue, sleep-related markers, sensory or olfactory function, biochemical markers, molecular markers, dopamine levels, α-synuclein, neuroinflammatory markers, oxidative stress markers, neurotrophic factors, pro-inflammatory cytokines, electrophysiological markers, and imaging-derived measures of dopaminergic function, basal ganglia structure, or functional connectivity, or a combination thereof.

25. A digital therapeutic system for modulating one or more Parkinson's disease-related markers, symptoms, or disease progression in an individual in need thereof, the system comprising:
a data-input module configured to onboard the individual and dynamically monitor and store data related to the individual throughout a therapeutic plan; and
a digital therapy delivery module comprising a processor configured to deliver the therapeutic plan comprising therapeutic interventions selected from: at least one motor skills intervention, at least one psychological intervention, at least one cognitive intervention, or a combination thereof;
wherein the digital therapy delivery module is further configured to convert the therapeutic plan into a plurality of targeted interaction outputs for presentation to the individual and track the individual's responses.

26. The system of claim 25 wherein the digital therapy delivery module comprises software executable on a personal electronic device.

27. The system of claim 25, wherein the data-input module is configured to receive data from a plurality of data acquisition sources in real-time, wherein the data comprises one or more of: Electronic Medical Records (EMR) data, motion sensor data, position sensor data, environmental sensors data, audio sensors data, location sensors data, audio recording data, speech recognition data, gaze-tracking data, touch interaction data, physiological sensor data, optical sensor, imaging sensors, self-report data, or a combination thereof.

28. The system of claim 25 wherein the digital therapy delivery module comprises any of the following selected from: a personal electronic device, a graphical user interface, audio interface, haptic interface, or a combination thereof.

29. The system of claim 25 wherein the processor is further configured to:
dynamically monitor any of the following factors of the individual selected from: Electronic Medical Records (EMR) data, motor attributes, psychological attributes, cognitive attributes, pharmacological attributes, and drug intake parameters, or a combination thereof, throughout the implementation of the therapeutic plan;
generate a dynamic user profile, and recursively adapt the therapeutic plan based on the dynamic user profile, performance metrics, and the individual's responses;
or a combination thereof.

30. The system of claim 25 further comprising at least one additional device operatively coupled to the digital therapy delivery module, selected from: health monitoring systems, medical devices, haptic devices, external speakers, headphones, virtual reality headsets, augmented reality glasses or devices, biofeedback sensors, wearable activity trackers, smartphones, tablets, smartwatches, personal computational devices, smart speakers, voice assistants, motion tracking sensors, virtual assistant systems, internet hubs, gait sensors, eye-tracking devices, voice recording devices, and wearable devices, or combinations thereof.
